(19) United States
(12) Patent Application Publication
(10) Pub. No.: US 2022/0265666 A1
(43) Pub. Date: Aug. 25, 2022

(54) METHODS OF TREATING SUBSTANCE ABUSE DISORDER, DYSPNEA, TINNITUS, AND CHILD AND ADOLESCENT DEPRESSION

(71) Applicant: Praxis Precision Medicines, Inc., Boston, MA (US)

(72) Inventors: Kiran REDDY, Charlestown, MA (US); Carlos LOYA, Cambridge, MA (US)

(21) Appl. No.: 17/632,172

(22) PCT Filed: Jul. 31, 2020
(86) PCT No.: PCT/US2020/044455

§ 371 Date: Feb. 1, 2022

Related U.S. Application Data
(60) Provisional application No. 62/882,321, filed on Aug. 2, 2019.

(51) Int. Cl. A61K 31/536

(52) U.S. Cl. CPC A61K 31/536

(57) ABSTRACT

The present invention relates to methods for the treatment of substance abuse disorder, dyspnea, tinnitus, child and adolescent depression, child and adolescent suicidal ideation and behavior, and child and adolescent anxiety using etifoxine or a pharmaceutically acceptable salt thereof.

### CROSS REFERENCE TO RELATED APPLICATIONS

This application claims priority to U.S. Application No. 62/882,321, filed Aug. 2, 2019, which is hereby incorporated by reference in its entirety herein.

### FIELD OF THE DISCLOSURE

The present disclosure relates to methods for the treatment of a substance abuse disorder, dyspnea, tinnitus, child and adolescent depression, child and adolescent suicidal ideation and behavior, and child and adolescent anxiety using etifoxine.

### BACKGROUND OF THE DISCLOSURE

Etifoxine is a non-benzodiazepine anxiolytic drug whose molecular target is the β2 and β3 subunit of the γ-aminobutyric acid type A (GABAA) receptor, where it modulates channel function. Etifoxine also increases the levels of endogenous neurosteroids and neuroactive steroids. The chemical name of etifoxine is 6-chloro-2-(ethylamino)-4-methyl-4-phenyl-4H-3, 1-benzoxazine and its structural formula is shown below.

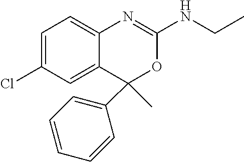 [SMILES: CCNC1=Nc2ccc(Cl)cc2C(C)(c2ccccc2)O1]

The hydrochloride salt of etifoxine is marketed as Stresam® in France and other markets for the treatment of anxiety. Stresam® is administered in 150 mg and up to 200 mg daily doses provided as one 50 mg tablet administered three times daily and two 50 mg tablets administered two times daily, respectively.

### SUMMARY OF THE DISCLOSURE

The present disclosure, among other things, provides methods of treating of a substance abuse disorder, dyspnea, and tinnitus by administering a therapeutically effective amount of etifoxine or a pharmaceutically acceptable salt thereof to a patient in need thereof.

In one aspect, the present disclosure provides methods of treating a substance abuse disorder comprising administering a therapeutically effective amount of etifoxine or a pharmaceutically acceptable salt thereof to a patient in need thereof. In some embodiments, the substance abuse disorder is opioid use disorder.

In one aspect, the present disclosure provides methods of treating dyspnea comprising administering a therapeutically effective amount of etifoxine or a pharmaceutically acceptable salt thereof to a patient in need thereof. In some embodiments, the dyspnea is selected from the group consisting of dyspnea in asthma, dyspnea in chronic obstructive pulmonary disorder (COPD), dyspnea in idiopathic pulmonary fibrosis, dyspnea in cancer and dyspnea in other severe respiratory disorders. In some embodiments, the dyspnea is dyspnea in heart disease (such as congestive heart failure). In some embodiments, the dyspnea is dyspnea in gastroesophageal reflux disease (GERD).

In one aspect, the present disclosure provides methods of treating tinnitus comprising administering a therapeutically effective amount of etifoxine or a pharmaceutically acceptable salt thereof to a patient in need thereof.

In some embodiments, the patient in need of a treatment of tinnitus is a patient with chronic tinnitus. In some embodiments, the patient in need of a treatment of tinnitus is a patient with chronic subjective tinnitus. In some embodiments, the patient in need of a treatment of tinnitus is a patient with acute peripheral tinnitus.

In one aspect, the present disclosure provides methods of treating child and adolescent depression comprising administering a therapeutically effective amount of etifoxine or a pharmaceutically acceptable salt thereof to a patient in need thereof. In some embodiments, the patient is treated during puberty. In some embodiments, the patient is treated during menarche or menarche transition. In some embodiments, the patient is treated during spermarche or spermarche transition.

In some aspect, the present disclosure provides methods of treating child and adolescent suicidal ideation and behavior by administering a therapeutically effective amount of etifoxine or a pharmaceutically acceptable salt thereof to a patient in need thereof. In some embodiments, the patient is treated during puberty. In some embodiments, the patient is treated during menarche or menarche transition. In some embodiments, the patient is treated during spermarche or spermarche transition.

In one aspect, the present disclosure provides methods of treating child and adolescent anxiety by administering a therapeutically effective amount of etifoxine or a pharmaceutically acceptable salt thereof to a patient in need thereof. In some embodiments, the patient is treated during puberty. In some embodiments, the patient is treated during menarche or menarche transition. In some embodiments, the patient is treated during spermarche or spermarche transition.

In some embodiments, the methods comprise administering a daily dose of about 1 mg to about 500 mg of etifoxine or a pharmaceutically acceptable salt thereof to a patient in need thereof. In certain embodiments, the methods comprise administering a daily dose of about 150 mg to about 200 mg. In some embodiments, the methods comprise administering a daily dose of about 50 mg, about 75 mg, about 100 mg, about 125 mg, about 150 mg, about 175 mg, about 200 mg, about 225 mg, about 250 mg, about 275 mg, or about 300 mg, about 325 mg, about 350 mg, about 375 mg, or about 400 mg, about 425 mg, about 450 mg, about 475 mg, or about 500 mg of etifoxine or a pharmaceutically acceptable salt thereof to a patient in need thereof. In certain embodiments, the methods comprise administering a daily dose of about 150 mg of etifoxine or a pharmaceutically acceptable salt thereof to a patient in need thereof. In certain embodiments, the methods comprise administering a daily dose of about 200 mg of etifoxine or a pharmaceutically acceptable salt thereof to a patient in need thereof. In certain embodiments, the daily dose of etifoxine is provided by oral administration.

In some embodiments, the etifoxine is administered on a once-a-day basis. In some embodiments, the etifoxine is administered on a twice-a-day basis. In some embodiments, the etifoxine is administered on a thrice-a-day basis.

### Definitions

The term “about” when immediately preceding a numerical value means a range (e.g., plus or minus 10% of that value). For example, “about 50” can mean 45 to 55. “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “acute tinnitus” as used herein refers to tinnitus that lasts less than 6 months.

The terms “administer,” “administering” or “administration” as used herein refer to either directly administering a compound or pharmaceutically acceptable salt or ester of the compound or a composition comprising the compound or pharmaceutically acceptable salt or ester of the compound to a patient.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical agent from one organ, or portion of the body, to another organ or portion of the body.

The term “child and adolescent depression” is used in this disclosure to mean child and adolescent depression as defined in the Diagnostic and Statistical Manual of Mental Disorders (DSM-5).

The term “child and adolescent suicidal ideation and behavior” is used in this disclosure to mean a child and adolescent with suicidal ideation and behavior as assessed by the Columbia-suicide severity rating scale (C-SSRS) and defined in the Diagnostic and Statistical Manual of Mental Disorders (DSM-5).

The phrase “puberty” as used herein refers to puberty as defined by a validated staging system. In some embodiments, “puberty” refers to puberty as defined by the Stages of Reproductive Aging Workshop 10 Staging System (for female patients). In some embodiments, “puberty” refers to puberty as defined by the Tanner Stages Staging System.

The phrase “spermarche” and “spermarche transition” as used herein refers to spermarche and spermarche transition, respectively, as defined by a validated staging system. In some embodiments, “spermarche” and “spermarche transition” as used herein refers to spermarche and spermarche transition, respectively, as defined by the Tanner Stages Staging System.

The phrase “menarche” and “menarche transition” as used herein refers to menarche and menarche transition, respectively, as defined by a validated staging system. In some embodiments, “menarche” and “menarche transition” as used herein refers to menarche and menarche transition, respectively, as defined by the Stages of Reproductive Aging Workshop 10 Staging System. In some embodiments, “menarche” and “menarche transition” as used herein refers to menarche and menarche transition, respectively, as defined by the Tanner Stages Staging System.

The term “chronic tinnitus” as used herein refers to tinnitus that lasts for 6 months or more.

The term “disorder” as used herein means, and is used interchangeably with, the terms disease, condition, or illness, unless otherwise indicated.

The terms “effective amount” and “therapeutically effective amount” are used interchangeably in this disclosure and refer to an amount of a compound, or a salt, solvate or ester thereof, that, when administered to a patient, is capable of performing the intended result. For example, in some embodiments, an effective amount of etifoxine is that amount that is required to reduce at least one symptom of tinnitus in a patient. The actual amount that comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid.

The term “pharmaceutically acceptable salts” includes those obtained by reacting the active compound functioning as a base, with an inorganic or organic acid to form a salt, for example, salts of 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid, (−L) malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, propionic acid, pyroglutamic acid (−L), salicylic acid, sebacic acid, stearic acid, succinic acid, sulfuric acid, tartaric acid (+L), thiocyanic acid, toluenesulfonic acid (p), and undecylenic acid. Those skilled in the art will further recognize that acid addition salts may be prepared by reaction of the compounds with the appropriate inorganic or organic acid via any of a number of known methods.

The term “severe respiratory disorder” as used herein means a respiratory disease or disorder wherein the patient exhibits abnormal pulmonary function tests where FVC and FEV1 are less than 80% of the reference value.

The term “tinnitus” as used herein is a perception of sound (for example, a buzzing, ringing, or hissing) in proximity to the head in the absence of an external source. Tinnitus as used herein includes subjective tinnitus, objective tinnitus, neurological tinnitus and somatic tinnitus. Tinnitus may be chronic or acute. Non-limiting examples of tinnitus as used herein include drug-induced tinnitus, tinnitus caused by an otologic condition, tinnitus caused by a vascular disease or disorder, tinnitus caused by a neurologic condition, tinnitus caused by an infectious condition, tinnitus caused by genetic predispositions, presbycusis, otosclerosis, endocrine or metabolic damage of the auditory system.

The term “treating” as used herein with regard to a patient, refers to improving at least one symptom of the patient's disorder. Treating can be curing, improving, or at least partially ameliorating a disorder.

The term “therapeutic effect” as used herein refers to a desired or beneficial effect provided by the method and/or the composition. For example, in some embodiments, the method for treating tinnitus provides a therapeutic effect when the method reduces at least one symptom of tinnitus in a patient.

The term “dyspnea” as used herein means a subjective experience of breathing discomfort that consists of qualitatively distinct sensations that vary in intensity.

### DETAILED DESCRIPTION OF THE DISCLOSURE

The present disclosure provides methods for the treatment of a substance abuse disorder; dyspnea; and tinnitus comprising administering a therapeutically effective amount of etifoxine or a pharmaceutically acceptable salt thereof.

### Etifoxine

Etifoxine as employed in the present methods can form a part of a pharmaceutical composition by combining etifoxine, or a pharmaceutically acceptable salt thereof, with a pharmaceutically acceptable carrier. Additionally, the compositions can include an additive selected from the group consisting of adjuvants, excipients, diluents, release-modifying agents and stabilizers. The composition can be an immediate release formulation, a delayed release formulation, a sustained release formulation or an extended release formulation.

Etifoxine is a TSPO agonist that increases neuroactive steroid synthesis and increases levels of allopregnanolone and other positive endogenously synthesized GABA-A positive allosteric modulator neuroactive steroids. Hence, etifoxine indirectly enhances GABA-A neurotransmission by increase of endogenous neuroactive steroid levels. Etifoxine (marketed by Biocodex under the trade name Stresam™) is approved in several countries to treat anxiety.

The synthesis of etifoxine is described in U.S. Pat. No. 3,725,404 and pharmaceutical compositions containing etifoxine are described in International Publication No. 2014/181280, which are hereby incorporated by reference in their entirety for all purposes. In some embodiments, the formulations and methods of the present disclosure utilize a pharmaceutical composition described in International Publication No. 2014/181280.

The synthesis of R-etifoxine is described in U.S. Publication No. 2008/039453; the synthesis of S-etifoxine is described in U.S. Pat. No. 8,110,569; and deuterated derivatives of etifoxine are described in U.S. Pat. No. 10,080,755, which are hereby incorporated by reference in their entirety for all purposes.

In some embodiments, the etifoxine used in the formulations and methods of the present disclosure is R-etifoxine or a pharmaceutically acceptable salt thereof. In some embodiments, the etifoxine used in the formulations and methods of the present disclosure is S-etifoxine or a pharmaceutically acceptable salt thereof. In some embodiments, the etifoxine used in the formulations and methods of the present disclosure is a deuterated derivative of etifoxine as described in U.S. Pat. No. 10,080,755.

In some embodiments, the etifoxine used in the formulations and methods of the present disclosure is a pharmaceutically acceptable salt of etifoxine. In some embodiments, the pharmaceutically acceptable salt of etifoxine used in the formulations and methods of the present disclosure is selected from the group consisting of hydrobromide, hydrochloride, hydrogensulfate, mesylate, succinate and tartrate. In certain embodiments, the salt of etifoxine is etifoxine hydrobromide. In certain embodiments, the salt of etifoxine is etifoxine hydrochloride. In certain embodiments, the salt of etifoxine is etifoxine hydrogensulfate. In certain embodiments, the salt of etifoxine is etifoxine succinate. In certain embodiments, the salt of etifoxine is etifoxine tartrate.

### Formulations

The methods of the present invention can employ various formulations for administration to patients, e.g., humans in unit dosage forms, such as tablets, capsules, pills, powders, granules, sterile parenteral solutions or suspensions (e.g., intramuscular (IM), subcutaneous (SC) and intravenous (IV)), transdermal patches, oral solutions or suspensions, and oil-water emulsions and otic solutions or suspensions and oil-water emulsions containing suitable quantities of etifoxine or a pharmaceutically acceptable salt thereof.

Oral pharmaceutical dosage forms can be either solid or liquid. The solid dosage forms can be tablets, capsules, granules, films (e.g., buccal films) and bulk powders. Types of oral tablets include compressed, chewable lozenges and tablets, which can be enteric-coated, sugar-coated or film-coated. Capsules can be hard or soft gelatin capsules, while granules and powders can be provided in non-effervescent or effervescent form with the combination of other ingredients known to those skilled in the art. In some embodiments, the present oral dosage forms may include orally disintegrating tablets.

Pharmaceutically acceptable carriers utilized in tablets include binders, lubricants, diluents, disintegrating agents, coloring agents, flavoring agents, and wetting agents.

Liquid oral dosage forms include aqueous solutions, emulsions, suspensions, solutions and/or suspensions reconstituted from non-effervescent granules and effervescent preparations reconstituted from effervescent granules.

Aqueous solutions include, for example, elixirs and syrups. Emulsions can be either oil-in water or water-in-oil. Elixirs are clear, sweetened, hydroalcoholic preparations. Pharmaceutically acceptable carriers used in elixirs include solvents. Syrups can be concentrated aqueous solutions of a sugar, for example, sucrose, and can contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions can use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substance used in effervescent granules, to be reconstituted into a liquid oral dosage form, can include organic acids and a source of carbon dioxide. Coloring and flavoring agents can be used in all of the above dosage forms.

Otic pharmaceutical dosage forms can be either gels or liquids, including aqueous and non-aqueous solutions, emulsions and suspensions. Pharmaceutically acceptable carriers used in otic pharmaceutical dosage forms are known to those skilled in the art and include pharmaceutically acceptable solvents, preservatives, stabilizers, wetting agents, emulsifiers, salts for regulating the osmotic pressure and buffers. In certain embodiments, the otic dosage form is a gel, which is biodegradable or non-biodegradable, aqueous or non-aqueous or microspheres are based. Examples of such gels and other suitable materials include poloxamers, Hyaluronate, xyloglucans, chitosan, polyesters, polylactide, polyglycolide and their copolymers PLGA polymer, poly anhydrides, poly caprolactone sucrose and glycerol monooleate. In certain embodiments, otic pharmaceutical dosage forms comprise penetration enhancers to facilitate the delivery of the composition across biological barriers that separate the middle and inner ear, e.g., the round window, thereby efficiently delivery a therapeutically effective amount of the composition to the inner car.

In some embodiments, the present disclosure provides a pharmaceutical composition comprising a salt of etifoxine. In some embodiments, the salt of etifoxine is etifoxine hydrobromide, etifoxine hydrochloride, etifoxine hydrogensulfate, etifoxine mesylate, etifoxine succinate and etifoxine tartrate.

In some embodiments, the pharmaceutical compositions described herein comprise etifoxine in an amount of from about 1 mg to about 500 mg, e.g., about 1 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg, including all values and subranges therebetween. In particular embodiments, the pharmaceutical compositions described herein comprise etifoxine in an amount of from about 50 mg to about 200 mg. In other particular embodiments, the pharmaceutical compositions described herein comprise about 50 mg, about 75 mg, about 100 mg, about 125 mg, about 150 mg, about 175 mg or about 200 mg of etifoxine.

### Administration and Dosing

The invention provides methods for treating a substance abuse disorder, dyspnea, tinnitus, child and adolescent depression, child and adolescent anxiety, and child and adolescent suicidal ideation and behavior, by administering an effective amount of etifoxine or a pharmaceutically acceptable salt thereof to a patient in need thereof.

In some embodiments, an effective amount is an amount sufficient to eliminate or significantly reduce substance abuse disorder symptoms or to alleviate those symptoms (e.g., reduce the amount of the patient's opioid use, compared to the symptoms present prior to treatment).

In some embodiments, an effective amount is an amount sufficient to eliminate or significantly reduce dyspnea symptoms or to alleviate those symptoms (e.g., reduce the amount of patient's breathlessness, compared to the symptoms present prior to treatment).

In some embodiments, an effective amount is an amount sufficient to eliminate or significantly reduce tinnitus symptoms or to alleviate those symptoms (e.g., reduce the symptoms, such as ear ringing, compared to the symptoms present prior to treatment).

In some embodiments, an effective amount is an amount sufficient to eliminate or significantly reduce child and adolescent depression, or child and adolescent anxiety, or child and adolescent suicidal ideation and behavior symptoms or to alleviate those symptoms (e.g., reduce the symptoms, such as frequency of suicidal ideation), compared to the symptoms present prior to treatment).

In some embodiments, the etifoxine is administered by otic administration. In certain embodiments, the etifoxine is administered auricular, intraauricular, intracochlear, intravestibular, or transtympanically, e.g., by injection. In some embodiments, administration is directly to the inner ear, e.g. injection through the round window, otic capsule, or vestibular canals. In some embodiments, administration is directly into the inner ear via a cochlear implant delivery system. In some embodiments, the otic formulation is injected transtympanically to the middle ear.

In some embodiments, the etifoxine is orally administered.

According to some embodiments of the present disclosure, administering the etifoxine compositions of the present disclosure provides a statistically significant therapeutic effect. In some embodiments, the statistically significant therapeutic effect is determined based on one or more standards or criteria provided by one or more regulatory agencies in the United States, e.g., FDA or other countries (such as Australia). In another embodiment, the statistically significant therapeutic effect is determined based on results obtained from regulatory agency approved clinical trial set up and/or procedure.

In some embodiments, the statistically significant therapeutic effect is determined based on a patient population of at least 20, 50, 60, 100, 200, 300, 400, 500, 600, 700, 800, 900, 1000 or 2000. In some embodiments, the statistically significant therapeutic effect is determined based on data obtained from randomized and double-blinded clinical trial set up. In some embodiments, the statistically significant therapeutic effect is determined based on data with a p value of less than or equal to about 0.05, 0.04, 0.03, 0.02 or 0.01. In some embodiments, the statistically significant therapeutic effect is determined based on data with a confidence interval greater than or equal to 95%, 96%, 97%, 98% or 99%.

In some embodiments, the statistically significant therapeutic effect is determined by a randomized double-blind clinical trial of patients treated with etifoxine or a pharmaceutically acceptable salt thereof and optionally in combination with the standard of care. The methods for determining a therapeutic effect will depend on the treated condition. Exemplary methods for measuring statistically significant effects for certain disorders (including opioid use disorder, dyspnea associated with chronic obstructive pulmonary disease, dyspnea associated with idiopathic pulmonary fibrosis, or dyspnea associated with asthma, and tinnitus) are described in the Methods of Treatment described of the present disclosure.

In general, statistical analysis can include any suitable method permitted by a regulatory agency, e.g., FDA in the US or Europe or any other country. In some embodiments, statistical analysis includes non-stratified analysis, log-rank analysis, e.g., from Kaplan-Meier, Jacobson-Truax, Gulliken-Lord-Novick, Edwards-Nunnally, Hageman-Arrindel and Hierarchical Linear Modeling (HLM) and Cox regression analysis.

In some embodiments, the doses provided herein refer to the amount of etifoxine a day that used to treat “a disease or condition described herein”. In the context of the present disclosure, such embodiments describe the recited daily dose for each of the conditions described in the present disclosure. For example, a total daily dose of etifoxine is at least about 5 mg a day for the treatment of a disease or condition described herein describes, for example, the treatment of substance abuse disorder, dyspnea, tinnitus, child and adolescent depression, child and adolescent anxiety, and child and adolescent suicidal ideation and behavior by administering a total daily dose of etifoxine of at least about 5 mg a day.

In some embodiments, a total daily dose of etifoxine is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, and about 250 mg.

In certain embodiments, the total daily dose of etifoxine is from about 5 mg to about 250 mg, including about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, and about 250 mg including all ranges there between. In certain embodiments, the total daily dose of etifoxine is from about 150 mg to about 200 mg.

In some embodiments, the total daily dose of etifoxine is at least about 5 mg a day for the treatment of a disease or condition described herein. In some embodiments, the total daily dose of etifoxine is at least about 10 mg a day for the treatment of a disease or condition described herein. In some embodiments, the total daily dose of etifoxine is at least about 15 mg a day for the treatment of a disease or condition described herein. In some embodiments, the total daily dose of etifoxine is at least about 20 mg a day for the treatment of a disease or condition described herein. In some embodiments, the total daily dose of etifoxine is at least about 25 mg a day for the treatment of a disease or condition described herein. In some embodiments, the total daily dose of etifoxine is at least about 30 mg a day for the treatment of a disease or condition described herein. In some embodiments, the total daily dose of etifoxine is at least about 35 mg a day for the treatment of a disease or condition described herein. In some embodiments, the total daily dose of etifoxine is at least about 40 mg a day for the treatment of a disease or condition described herein. In some embodiments, the total daily dose of etifoxine is at least about 45 mg a day for the treatment of a disease or condition described herein. In some embodiments, the total daily dose of etifoxine is at least about 50 mg a day for the treatment of a disease or condition described herein. In some embodiments, the total daily dose of etifoxine is at least about 55 mg a day for the treatment of a disease or condition described herein. In some embodiments, the total daily dose of etifoxine is at least about 60 mg a day for the treatment of a disease or condition described herein. In some embodiments, the total daily dose of etifoxine is at least about 65 mg a day for the treatment of a disease or condition described herein. In some embodiments, the total daily dose of etifoxine is at least about 70 mg a day for the treatment of a disease or condition described herein. In some embodiments, the total daily dose of etifoxine is at least about 75 mg a day for the treatment of a disease or condition described herein. In some embodiments, the total daily dose of etifoxine is at least about 80 mg a day for the treatment of a disease or condition described herein. In some embodiments, the total daily dose of etifoxine is at least about 85 mg a day for the treatment of a disease or condition described herein. In some embodiments, the total daily dose of etifoxine is at least about 90 mg a day for the treatment of a disease or condition described herein. In some embodiments, the total daily dose of etifoxine is at least about 95 mg a day for the treatment of a disease or condition described herein. In some embodiments, the total daily dose of etifoxine is at least about 100 mg a day for the treatment of a disease or condition described herein. In some embodiments, the total daily dose of etifoxine is at least about 105 mg a day for the treatment of a disease or condition described herein. In some embodiments, the total daily dose of etifoxine is at least about 110 mg a day for the treatment of a disease or condition described herein. In some embodiments, the total daily dose of etifoxine is at least about 115 mg a day for the treatment of a disease or condition described herein. In some embodiments, the total daily dose of etifoxine is at least about 120 mg a day for the treatment of a disease or condition described herein. In some embodiments, the total daily dose of etifoxine is at least about 125 mg a day for the treatment of a disease or condition described herein. In some embodiments, the total daily dose of etifoxine is at least about 130 mg a day for the treatment of a disease or condition described herein. In some embodiments, the total daily dose of etifoxine is at least about 135 mg a day for the treatment of a disease or condition described herein. In some embodiments, the total daily dose of etifoxine is at least about 140 mg a day for the treatment of a disease or condition described herein. In some embodiments, the total daily dose of etifoxine is at least about 145 mg a day for the treatment of a disease or condition described herein. In some embodiments, the total daily dose of etifoxine is at least about 150 mg a day for the treatment of a disease or condition described herein. In some embodiments, the total daily dose of etifoxine is at least about 155 mg a day for the treatment of a disease or condition described herein. In some embodiments, the total daily dose of etifoxine is at least about 160 mg a day for the treatment of a disease or condition described herein. In some embodiments, the total daily dose of etifoxine is at least about 165 mg a day for the treatment of a disease or condition described herein. In some embodiments, the total daily dose of etifoxine is at least about 170 mg a day for the treatment of a disease or condition described herein. In some embodiments, the total daily dose of etifoxine is at least about 175 mg a day for the treatment of a disease or condition described herein. In some embodiments, the total daily dose of etifoxine is at least about 180 mg a day for the treatment of a disease or condition described herein. In some embodiments, the total daily dose of etifoxine is at least about 185 mg a day for the treatment of a disease or condition described herein. In some embodiments, the total daily dose of etifoxine is at least about 190 mg a day for the treatment of a disease or condition described herein. In some embodiments, the total daily dose of etifoxine is at least about 195 mg a day for the treatment of a disease or condition described herein. In some embodiments, the total daily dose of etifoxine is at least about 200 mg a day for the treatment of a disease or condition described herein. In some embodiments, the total daily dose of etifoxine is at least about 205 mg a day for the treatment of a disease or condition described herein. In some embodiments, the total daily dose of etifoxine is at least about 210 mg a day for the treatment of a disease or condition described herein. In some embodiments, the total daily dose of etifoxine is at least about 215 mg a day for the treatment of a disease or condition described herein. In some embodiments, the total daily dose of etifoxine is at least about 220 mg a day for the treatment of a disease or condition described herein. In some embodiments, the total daily dose of etifoxine is at least about 225 mg a day for the treatment of a disease or condition described herein. In some embodiments, the total daily dose of etifoxine is at least about 230 mg a day for the treatment of a disease or condition described herein. In some embodiments, the total daily dose of etifoxine is at least about 235 mg a day for the treatment of a disease or condition described herein. In some embodiments, the total daily dose of etifoxine is at least about 240 mg a day for the treatment of a disease or condition described herein. In some embodiments, the total daily dose of etifoxine is at least about 245 mg a day for the treatment of a disease or condition described herein. In some embodiments, the total daily dose of etifoxine is at least about 250 mg a day for the treatment of a disease or condition described herein.

In some embodiments, the total daily dose of etifoxine is about 5 mg a day for the treatment of a disease or condition described herein. In some embodiments, the total daily dose of etifoxine is about 10 mg a day for the treatment of a disease or condition described herein. In some embodiments, the total daily dose of etifoxine is about 15 mg a day for the treatment of a disease or condition described herein. In some embodiments, the total daily dose of etifoxine is about 20 mg a day for the treatment of a disease or condition described herein. In some embodiments, the total daily dose of etifoxine is about 25 mg a day for the treatment of a disease or condition described herein. In some embodiments, the total daily dose of etifoxine is about 30 mg a day for the treatment of a disease or condition described herein. In some embodiments, the total daily dose of etifoxine is about 35 mg a day for the treatment of a disease or condition described herein. In some embodiments, the total daily dose of etifoxine is about 40 mg a day for the treatment of a disease or condition described herein. In some embodiments, the total daily dose of etifoxine is about 45 mg a day for the treatment of a disease or condition described herein. In some embodiments, the total daily dose of etifoxine is about 50 mg a day for the treatment of a disease or condition described herein. In some embodiments, the total daily dose of etifoxine is about 55 mg a day for the treatment of a disease or condition described herein. In some embodiments, the total daily dose of etifoxine is about 60 mg a day for the treatment of a disease or condition described herein. In some embodiments, the total daily dose of etifoxine is about 65 mg a day for the treatment of a disease or condition described herein. In some embodiments, the total daily dose of etifoxine is about 70 mg a day for the treatment of a disease or condition described herein. In some embodiments, the total daily dose of etifoxine is about 75 mg a day for the treatment of a disease or condition described herein. In some embodiments, the total daily dose of etifoxine is about 80 mg a day for the treatment of a disease or condition described herein. In some embodiments, the total daily dose of etifoxine is about 85 mg a day for the treatment of a disease or condition described herein. In some embodiments, the total daily dose of etifoxine is about 90 mg a day for the treatment of a disease or condition described herein. In some embodiments, the total daily dose of etifoxine is about 95 mg a day for the treatment of a disease or condition described herein. In some embodiments, the total daily dose of etifoxine is about 100 mg a day for the treatment of a disease or condition described herein. In some embodiments, the total daily dose of etifoxine is about 105 mg a day for the treatment of a disease or condition described herein. In some embodiments, the total daily dose of etifoxine is about 110 mg a day for the treatment of a disease or condition described herein. In some embodiments, the total daily dose of etifoxine is about 115 mg a day for the treatment of a disease or condition described herein. In some embodiments, the total daily dose of etifoxine is about 120 mg a day for the treatment of a disease or condition described herein. In some embodiments, the total daily dose of etifoxine is about 125 mg a day for the treatment of a disease or condition described herein. In some embodiments, the total daily dose of etifoxine is about 130 mg a day for the treatment of a disease or condition described herein. In some embodiments, the total daily dose of etifoxine is about 135 mg a day for the treatment of a disease or condition described herein. In some embodiments, the total daily dose of etifoxine is about 140 mg a day for the treatment of a disease or condition described herein. In some embodiments, the total daily dose of etifoxine is about 145 mg a day for the treatment of a disease or condition described herein. In some embodiments, the total daily dose of etifoxine is about 150 mg a day for the treatment of a disease or condition described herein. In some embodiments, the total daily dose of etifoxine is about 155 mg a day for the treatment of a disease or condition described herein. In some embodiments, the total daily dose of etifoxine is about 160 mg a day for the treatment of a disease or condition described herein. In some embodiments, the total daily dose of etifoxine is about 165 mg a day for the treatment of a disease or condition described herein. In some embodiments, the total daily dose of etifoxine is about 170 mg a day for the treatment of a disease or condition described herein. In some embodiments, the total daily dose of etifoxine is about 175 mg a day for the treatment of a disease or condition described herein. In some embodiments, the total daily dose of etifoxine is about 180 mg a day for the treatment of a disease or condition described herein. In some embodiments, the total daily dose of etifoxine is about 185 mg a day for the treatment of a disease or condition described herein. In some embodiments, the total daily dose of etifoxine is about 190 mg a day for the treatment of a disease or condition described herein. In some embodiments, the total daily dose of etifoxine is about 195 mg a day for the treatment of a disease or condition described herein. In some embodiments, the total daily dose of etifoxine is about 200 mg a day for the treatment of a disease or condition described herein. In some embodiments, the total daily dose of etifoxine is about 205 mg a day for the treatment of a disease or condition described herein. In some embodiments, the total daily dose of etifoxine is about 210 mg a day for the treatment of a disease or condition described herein. In some embodiments, the total daily dose of etifoxine is about 215 mg a day for the treatment of a disease or condition described herein. In some embodiments, the total daily dose of etifoxine is about 220 mg a day for the treatment of a disease or condition described herein. In some embodiments, the total daily dose of etifoxine is about 225 mg a day for the treatment of a disease or condition described herein. In some embodiments, the total daily dose of etifoxine is about 230 mg a day for the treatment of a disease or condition described herein. In some embodiments, the total daily dose of etifoxine is about 235 mg a day for the treatment of a disease or condition described herein. In some embodiments, the total daily dose of etifoxine is about 240 mg a day for the treatment of a disease or condition described herein. In some embodiments, the total daily dose of etifoxine is about 245 mg a day for the treatment of a disease or condition described herein. In some embodiments, the total daily dose of etifoxine is about 250 mg a day for the treatment of a disease or condition described herein.

In some embodiments, about 5 mg of etifoxine once a day (or twice a day, or three times a day) is selected to provide a substantial reduction in a disease or condition described herein. In some embodiments, about 10 mg of etifoxine once a day (or twice a day, or three times a day) is selected to provide a substantial reduction in a disease or condition described herein. In some embodiments, about 15 mg of etifoxine once a day (or twice a day, or three times a day) is selected to provide a substantial reduction in a disease or condition described herein. In some embodiments, about 20 mg of etifoxine once a day (or twice a day, or three times a day) is selected to provide a substantial reduction in a disease or condition described herein. In some embodiments, about 25 mg of etifoxine once a day (or twice a day, or three times a day) is selected to provide a substantial reduction in a disease or condition described herein. In some embodiments, about 30 mg of etifoxine once a day (or twice a day, or three times a day) is selected to provide a substantial reduction in a disease or condition described herein. In some embodiments, about 35 mg of etifoxine once a day (or twice a day, or three times a day) is selected to provide a substantial reduction in a disease or condition described herein. In some embodiments, about 40 mg of etifoxine once a day (or twice a day, or three times a day) is selected to provide a substantial reduction in a disease or condition described herein. In some embodiments, about 45 mg of etifoxine once a day (or twice a day, or three times a day) is selected to provide a substantial reduction in a disease or condition described herein. In some embodiments, about 50 mg of etifoxine once a day (or twice a day, or three times a day) is selected to provide a substantial reduction in a disease or condition described herein. In some embodiments, about 55 mg of etifoxine once a day (or twice a day, or three times a day) is selected to provide a substantial reduction in a disease or condition described herein. In some embodiments, about 60 mg of etifoxine once a day (or twice a day, or three times a day) is selected to provide a substantial reduction in a disease or condition described herein. In some embodiments, about 65 mg of etifoxine once a day (or twice a day, or three times a day) is selected to provide a substantial reduction in a disease or condition described herein. In some embodiments, about 70 mg of etifoxine once a day (or twice a day, or three times a day) is selected to provide a substantial reduction in a disease or condition described herein. In some embodiments, about 75 mg of etifoxine once a day (or twice a day, or three times a day) is selected to provide a substantial reduction in a disease or condition described herein. In some embodiments, about 80 mg of etifoxine once a day (or twice a day, or three times a day) is selected to provide a substantial reduction in a disease or condition described herein. In some embodiments, about 85 mg of etifoxine once a day (or twice a day) is selected to provide a substantial reduction in a disease or condition described herein. In some embodiments, about 90 mg of etifoxine once a day (or twice a day) is selected to provide a substantial reduction in a disease or condition described herein. In some embodiments, about 95 mg of etifoxine once a day (or twice a day) is selected to provide a substantial reduction in a disease or condition described herein. In some embodiments, about 100 mg of etifoxine once a day (or twice a day) is selected to provide a substantial reduction in a disease or condition described herein. In some embodiments, about 105 mg of etifoxine once a day (or twice a day) is selected to provide a substantial reduction in a disease or condition described herein. In some embodiments, about 110 mg of etifoxine once a day (or twice a day) is selected to provide a substantial reduction in a disease or condition described herein. In some embodiments, about 115 mg of etifoxine once a day (or twice a day) is selected to provide a substantial reduction in a disease or condition described herein. In some embodiments, about 120 mg of etifoxine once a day (or twice a day) is selected to provide a substantial reduction in a disease or condition described herein. In some embodiments, about 125 mg of etifoxine once a day (or twice a day) is selected to provide a substantial reduction in a disease or condition described herein. In some embodiments, about 130 mg of etifoxine once a day is selected to provide a substantial reduction in a disease or condition described herein. In some embodiments, about 135 mg of etifoxine once a day is selected to provide a substantial reduction in a disease or condition described herein. In some embodiments, about 140 mg of etifoxine once a day is selected to provide a substantial reduction in a disease or condition described herein. In some embodiments, about 145 mg of etifoxine once a day is selected to provide a substantial reduction in a disease or condition described herein. In some embodiments, about 150 mg of etifoxine once a day is selected to provide a substantial reduction in a disease or condition described herein. In some embodiments, about 155 mg of etifoxine once a day is selected to provide a substantial reduction in a disease or condition described herein. In some embodiments, about 160 mg of etifoxine once a day is selected to provide a substantial reduction in a disease or condition described herein. In some embodiments, about 165 mg of etifoxine once a day is selected to provide a substantial reduction in a disease or condition described herein. In some embodiments, about 170 mg of etifoxine once a day is selected to provide a substantial reduction in a disease or condition described herein. In some embodiments, about 175 mg of etifoxine once a day is selected to provide a substantial reduction in a disease or condition described herein. In some embodiments, about 180 mg of etifoxine once a day is selected to provide a substantial reduction in a disease or condition described herein. In some embodiments, about 185 mg of etifoxine once a day is selected to provide a substantial reduction in a disease or condition described herein. In some embodiments, about 190 mg of etifoxine once a day is selected to provide a substantial reduction in a disease or condition described herein. In some embodiments, about 195 mg of etifoxine once a day is selected to provide a substantial reduction in a disease or condition described herein. In some embodiments, about 200 mg of etifoxine once a day is selected to provide a substantial reduction in a disease or condition described herein. In some embodiments, about 205 mg of etifoxine once a day is selected to provide a substantial reduction in a disease or condition described herein. In some embodiments, about 210 mg of etifoxine once a day is selected to provide a substantial reduction in a disease or condition described herein. In some embodiments, about 215 mg of etifoxine once a day is selected to provide a substantial reduction in a disease or condition described herein. In some embodiments, about 220 mg of etifoxine once a day is selected to provide a substantial reduction in a disease or condition described herein. In some embodiments, about 225 mg of etifoxine once a day is selected to provide a substantial reduction in a disease or condition described herein. In some embodiments, about 230 mg of etifoxine once a day is selected to provide a substantial reduction in a disease or condition described herein. In some embodiments, about 235 mg of etifoxine once a day is selected to provide a substantial reduction in a disease or condition described herein. In some embodiments, about 240 mg of etifoxine once a day is selected to provide a substantial reduction in a disease or condition described herein. In some embodiments, about 245 mg of etifoxine once a day is selected to provide a substantial reduction in a disease or condition described herein. In some embodiments, about 250 mg of etifoxine once a day is selected to provide a substantial reduction in a disease or condition described herein.

In certain embodiments, etifoxine or a pharmaceutically acceptable salt thereof is administered on a once a day or twice a day or three times a day basis for at least about one day, for example, about 1 day, about 2 days, about 3 days, about 4 days, about 5 days, about 6 days, about 7 days, about 8 days, about 9 days, about 10 days, about 12 days, about 13 days and about 14 days.

In certain embodiments, etifoxine or a pharmaceutically acceptable salt thereof is administered on a once a day or twice a day or three times a day basis for at least a week, for example, about a week, about 2 weeks, about 3 weeks, about 4 weeks, about 5 weeks, about 6 weeks, about 7 weeks, about 8 weeks, about 9 weeks, about 10 weeks, about 12 weeks, about 18 weeks, about 24 weeks, and about 50 weeks.

In certain embodiments, at least about 5 mg or about 5 mg of etifoxine or a pharmaceutically acceptable salt thereof is administered on a once a day or twice a day or thrice a day basis for at least a week. In certain embodiments, at least about 10 mg or about 10 mg of etifoxine or a pharmaceutically acceptable salt thereof is administered on a once a day or twice a day or thrice a day basis for at least a week. In certain embodiments, at least about 15 mg or about 15 mg of etifoxine or a pharmaceutically acceptable salt thereof is administered on a once a day or twice a day or thrice a day basis for at least a week. In certain embodiments, at least about 20 mg or about 20 mg of etifoxine or a pharmaceutically acceptable salt thereof is administered on a once a day or twice a day or thrice a day basis for at least a week. In certain embodiments, at least about 25 mg or about 25 mg of etifoxine or a pharmaceutically acceptable salt thereof is administered on a once a day or twice a day or thrice a day basis for at least a week. In certain embodiments, at least about 30 mg or about 30 mg of etifoxine or a pharmaceutically acceptable salt thereof is administered on a once a day or twice a day or thrice a day basis for at least a week. In certain embodiments, at least about 35 mg or about 35 mg of etifoxine or a pharmaceutically acceptable salt thereof is administered on a once a day or twice a day or thrice a day basis for at least a week. In certain embodiments, at least about 40 mg or about 40 mg of etifoxine or a pharmaceutically acceptable salt thereof is administered on a once a day or twice a day or thrice a day basis for at least a week. In certain embodiments, at least about 45 mg or about 45 mg of etifoxine or a pharmaceutically acceptable salt thereof is administered on a once a day or twice a day or thrice a day basis for at least a week. In certain embodiments, at least about 50 mg or about 50 mg of etifoxine or a pharmaceutically acceptable salt thereof is administered on a once a day or twice a day or thrice a day basis for at least a week. In certain embodiments, at least about 55 mg or about 55 mg of etifoxine or a pharmaceutically acceptable salt thereof is administered on a once a day or twice a day or thrice a day basis for at least a week. In certain embodiments, at least about 60 mg or about 60 mg of etifoxine or a pharmaceutically acceptable salt thereof is administered on a once a day or twice a day or thrice a day basis for at least a week. In certain embodiments, at least about 65 mg or about 65 mg of etifoxine or a pharmaceutically acceptable salt thereof is administered on a once a day or twice a day or thrice a day basis for at least a week. In certain embodiments, at least about 70 mg or about 70 mg of etifoxine or a pharmaceutically acceptable salt thereof is administered on a once a day or twice a day or thrice a day basis for at least a week. In certain embodiments, at least about 75 mg or about 75 mg of etifoxine or a pharmaceutically acceptable salt thereof is administered on a once a day or twice a day or thrice a day basis for at least a week. In certain embodiments, at least about 80 mg or about 80 mg of etifoxine or a pharmaceutically acceptable salt thereof is administered on a once a day or twice a day or thrice a day basis for at least a week. In certain embodiments, at least about 85 mg or about 85 mg of etifoxine or a pharmaceutically acceptable salt thereof is administered on a once a day or twice a day basis for at least a week. In certain embodiments, at least about 90 mg or about 90 mg of etifoxine or a pharmaceutically acceptable salt thereof is administered on a once a day or twice a day basis for at least a week. In certain embodiments, at least about 95 mg or about 95 mg of etifoxine or a pharmaceutically acceptable salt thereof is administered on a once a day or twice a day basis for at least a week. In certain embodiments, at least about 100 mg or about 100 mg of etifoxine or a pharmaceutically acceptable salt thereof is administered on a once a day or twice a day basis for at least a week. In certain embodiments, at least about 105 mg or about 105 mg of etifoxine or a pharmaceutically acceptable salt thereof is administered on a once a day or twice a day basis for at least a week. In certain embodiments, at least about 110 mg or about 110 mg of etifoxine or a pharmaceutically acceptable salt thereof is administered on a once a day or twice a day basis for at least a week. In certain embodiments, at least about 115 mg or about 115 mg of etifoxine or a pharmaceutically acceptable salt thereof is administered on a once a day or twice a day basis for at least a week. In certain embodiments, at least about 120 mg or about 120 mg of etifoxine or a pharmaceutically acceptable salt thereof is administered on a once a day or twice a day basis for at least a week. In certain embodiments, at least about 125 mg or about 125 mg of etifoxine or a pharmaceutically acceptable salt thereof is administered on a once a day or twice a day for at least a week. In certain embodiments, at least about 130 mg or about 130 mg of etifoxine or a pharmaceutically acceptable salt thereof is administered on a once a day basis for at least a week. In certain embodiments, at least about 135 mg or about 135 mg of etifoxine or a pharmaceutically acceptable salt thereof is administered on a once a day basis for at least a week. In certain embodiments, at least about 140 mg or about 140 mg of etifoxine or a pharmaceutically acceptable salt thereof is administered on a once a day basis for at least a week. In certain embodiments, at least about 145 mg or about 145 mg of etifoxine or a pharmaceutically acceptable salt thereof is administered on a once a day basis for at least a week. In certain embodiments, at least about 150 mg or about 150 mg of etifoxine or a pharmaceutically acceptable salt thereof is administered on a once a day basis for at least a week. In certain embodiments, at least about 155 mg or about 155 mg of etifoxine or a pharmaceutically acceptable salt thereof is administered on a once a day basis for at least a week. In certain embodiments, at least about 160 mg or about 160 mg of etifoxine or a pharmaceutically acceptable salt thereof is administered on a once a day basis for at least a week. In certain embodiments, at least about 165 mg or about 165 mg of etifoxine or a pharmaceutically acceptable salt thereof is administered on a once a day basis for at least a week. In certain embodiments, at least about 170 mg or about 170 mg of etifoxine or a pharmaceutically acceptable salt thereof is administered on a once a day basis for at least a week. In certain embodiments, at least about 175 mg or about 175 mg of etifoxine or a pharmaceutically acceptable salt thereof is administered on a once a day basis for at least a week. In certain embodiments, at least about 180 mg or about 180 mg of etifoxine or a pharmaceutically acceptable salt thereof is administered on a once a day basis for at least a week. In certain embodiments, at least about 185 mg or about 185 mg of etifoxine or a pharmaceutically acceptable salt thereof is administered on a once a day basis for at least a week. In certain embodiments, at least about 190 mg or about 190 mg of etifoxine or a pharmaceutically acceptable salt thereof is administered on a once a day basis for at least a week. In certain embodiments, at least about 195 mg or about 195 mg of etifoxine or a pharmaceutically acceptable salt thereof is administered on a once a day basis for at least a week. In certain embodiments, at least about 200 mg or about 200 mg of etifoxine or a pharmaceutically acceptable salt thereof is administered on a once a day basis for at least a week. In certain embodiments, at least about 205 mg or about 205 mg of etifoxine or a pharmaceutically acceptable salt thereof is administered on a once a day basis for at least a week. In certain embodiments, at least about 210 mg or about 210 mg of etifoxine or a pharmaceutically acceptable salt thereof is administered on a once a day basis for at least a week. In certain embodiments, at least about 215 mg or about 215 mg of etifoxine or a pharmaceutically acceptable salt thereof is administered on a once a day basis for at least a week. In certain embodiments, at least about 220 mg or about 220 mg of etifoxine or a pharmaceutically acceptable salt thereof is administered on a once a day basis for at least a week. In certain embodiments, at least about 225 mg or about 225 mg of etifoxine or a pharmaceutically acceptable salt thereof is administered on a once a day basis for at least a week. In certain embodiments, at least about 230 mg or about 230 mg of etifoxine or a pharmaceutically acceptable salt thereof is administered on a once a day basis for at least a week. In certain embodiments, at least about 235 mg or about 235 mg of etifoxine or a pharmaceutically acceptable salt thereof is administered on a once a day basis for at least a week. In certain embodiments, at least about 240 mg or about 240 mg of etifoxine or a pharmaceutically acceptable salt thereof is administered on a once a day basis for at least a week. In certain embodiments, at least about 245 mg or about 245 mg of etifoxine or a pharmaceutically acceptable salt thereof is administered on a once a day basis for at least a week. In certain embodiments, at least about 250 mg or about 250 mg of etifoxine or a pharmaceutically acceptable salt thereof is administered on a once a day basis for at least a week.

In some embodiments, the present methods provide steady state plasma levels of etifoxine that correlate to one or more statistically significant therapeutic effects. In certain embodiments, the therapeutically effective steady state plasma levels of etifoxine provided by the methods of the present invention range from about 1 ng/mL to about 200 ng/mL, including about 1 ng/ml, about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL about 80 ng/mL, about 85 ng/mL, about 90 ng/mL, about 95 ng/mL, about 100 ng/mL, about 105 ng/mL, about 110 ng/mL, about 115 ng/mL, about 120 ng/mL, about 125 ng/mL, about 130 ng/mL, about 135 ng/mL, about 140 ng/mL, about 145 ng/mL, about 150 ng/mL, about 155 ng/mL, about 160 ng/mL, about 165 ng/mL, about 170 ng/mL, about 175 ng/mL about 180 ng/mL, about 185 ng/mL, about 190 ng/mL, about 195 ng/mL, and 200 ng/ml, including all ranges there between. In certain embodiments, the therapeutically effective steady state plasma levels of etifoxine provided by the methods of the present invention range from about 50 ng/ml to 200 ng/ml.

In certain embodiments, the therapeutically effective steady state plasma levels of Etifoxine is provided by administering a daily dose of Etifoxine or a pharmaceutically acceptable salt thereof of about 150 mg. In some further embodiments, the therapeutically effective steady state plasma levels of Etifoxine is provided by administering about 150 mg of Etifoxine or a pharmaceutically acceptable salt thereof once a day. In other further embodiments, the therapeutically effective steady state plasma levels of Etifoxine is provided by administering about 75 mg of Etifoxine or a pharmaceutically acceptable salt thereof twice a day. In other further embodiments, the therapeutically effective steady state plasma levels of Etifoxine is provided by administering about 50 mg of Etifoxine or a pharmaceutically acceptable salt thereof thrice a day.

In certain embodiments, the therapeutically effective steady state plasma levels of Etifoxine is provided by administering a daily dose of Etifoxine or a pharmaceutically acceptable salt thereof of about 200 mg. In some further embodiments, the therapeutically effective steady state plasma levels of Etifoxine is provided by administering about 200 mg of Etifoxine or a pharmaceutically acceptable salt thereof once a day. In other further embodiments, the therapeutically effective steady state plasma levels of Etifoxine is provided by administering about 100 mg of Etifoxine or a pharmaceutically acceptable salt thereof twice a day.

In some embodiments, the present methods provide mean steady state AUC_{0-24 h} (expressed in terms of ng*hr/mL) levels of etifoxine that correlate to one or more statistically significant therapeutic effects. In certain embodiments, the therapeutically effective mean steady state AUC_{0-24 h} levels of etifoxine provided by the methods of the present invention range from about 50 ng*hr/mL to about 2300 ng*hr/mL, including about 50 ng*hr/mL, 100 ng*hr/mL, 150 ng*hr/mL, 200 ng*hr/mL, 250 ng*hr/mL, 300 ng*hr/mL, about 400 ng*hr/mL, about 500 ng*hr/mL, about 600 ng*hr/mL, about 700 ng*hr/mL, about 800 ng*hr/mL, about 900 ng*hr/mL, about 1000 ng*hr/mL, about 1100 ng*hr/mL, about 1200 ng*hr/mL, about 1300 ng*hr/mL, about 1400 ng*hr/mL, about 1500 ng*hr/mL, about 1600 ng*hr/mL, about 1700 ng*hr/mL, about 1800 ng*hr/mL, about 1900 ng*hr/mL, about 2000 ng*hr/mL, about 2100 ng*hr/mL, about 2200 ng*hr/mL and about 2300 ng*hr/mL, including all ranges there between.

In certain embodiments, the therapeutically effective mean steady state AUC_{0-24 h} levels of Etifoxine is provided by administering a daily dose of Etifoxine or a pharmaceutically acceptable salt thereof of about 150 mg. In some further embodiments, the therapeutically effective mean steady state AUC_{0-24 h} levels of Etifoxine is provided by administering about 150 mg of Etifoxine or a pharmaceutically acceptable salt thereof once a day. In other further embodiments, the therapeutically effective mean steady state AUC_{0-24 h} levels of Etifoxine is provided by administering about 75 mg of Etifoxine or a pharmaceutically acceptable salt thereof twice a day. In other further embodiments, the therapeutically effective mean steady state AUC_{0-24 h} levels of Etifoxine is provided by administering about 50 mg of Etifoxine or a pharmaceutically acceptable salt thereof thrice a day.

In certain embodiments, the therapeutically effective mean steady state AUC_{0-24 h} levels of Etifoxine is provided by administering a daily dose of Etifoxine or a pharmaceutically acceptable salt thereof of about 200 mg. In some further embodiments, the therapeutically effective mean steady state AUC_{0-24 h} levels of Etifoxine is provided by administering about 200 mg of Etifoxine or a pharmaceutically acceptable salt thereof once a day. In other further embodiments, the therapeutically effective mean steady state AUC_{0-24 h} levels of Etifoxine is provided by administering about 100 mg of Etifoxine or a pharmaceutically acceptable salt thereof twice a day.

In some embodiments, the present methods provide steady state plasma Cmax levels of etifoxine that correlate to one or more statistically significant therapeutic effects. In certain embodiments, the therapeutically effective steady state plasma Cmax levels of etifoxine provided by the methods of the present invention range from about 5 ng/mL to about 500 ng/mL, including about 5 ng/mL, 10 ng/mL, 20 ng/mL, 30 ng/mL, 40 ng/mL, 50 ng/mL, 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, about 370 ng/mL about 380 ng/mL, about 390 ng/mL, about 400 ng/mL, about 410 ng/mL, about 420 ng/mL, about 430 ng/mL, about 440 ng/mL, about 150 ng/mL, about 460 ng/mL, about 470 ng/mL about 480 ng/mL, about 490 ng/mL, about 400 ng/mL, and about 500 ng/mL, including all ranges there between.

In certain embodiments, the therapeutically effective steady state plasma Cmax levels of Etifoxine is provided by administering a daily dose of Etifoxine or a pharmaceutically acceptable salt thereof of about 150 mg. In some further embodiments, the therapeutically effective steady state plasma Cmax levels of Etifoxine is provided by administering about 150 mg of Etifoxine or a pharmaceutically acceptable salt thereof once a day. In other further embodiments, the therapeutically effective steady state plasma Cmax levels of Etifoxine is provided by administering about 75 mg of Etifoxine or a pharmaceutically acceptable salt thereof twice a day. In other further embodiments, the therapeutically effective steady state plasma Cmax levels of Etifoxine is provided by administering about 50 mg of Etifoxine or a pharmaceutically acceptable salt thereof thrice a day.

In certain embodiments, the therapeutically effective steady state plasma Cmax levels of Etifoxine is provided by administering a daily dose of Etifoxine or a pharmaceutically acceptable salt thereof of about 200 mg. In some further embodiments, the therapeutically effective steady state plasma Cmax levels of Etifoxine is provided by administering about 200 mg of Etifoxine or a pharmaceutically acceptable salt thereof once a day. In other further embodiments, the therapeutically effective steady state plasma Cmax levels of Etifoxine is provided by administering about 100 mg of Etifoxine or a pharmaceutically acceptable salt thereof twice a day.

### Methods of Treatment:

In one aspect, the present disclosure provides methods of treating a condition selected from substance abuse disorder (such as opioid use disorder), dyspnea (such as dyspnea associated with chronic obstructive pulmonary disease, dyspnea associated with idiopathic pulmonary fibrosis, dyspnea associated with asthma), tinnitus, child and adolescent depression, child and adolescent anxiety, and child and adolescent suicidal ideation and behavior. The method comprises administering etifoxine or a pharmaceutically acceptable salt thereof to the patient in need of treatment. In some embodiments, the etifoxine or pharmaceutically acceptable salt thereof is administered in a pharmaceutical composition comprising etifoxine or pharmaceutically acceptable salt thereof. In some embodiments, the composition is administered on a once-a-day basis. In some embodiments, the composition is administered on a twice-a-day basis. In some embodiments, the composition is administered on a thrice-a-day basis.

Methods of Treating Substance Abuse Use Disorder

In some embodiments, the present disclosure provides methods of treating a substance abuse disorder (such as opioid use disorder) comprising administering an effective amount of etifoxine or a pharmaceutically acceptable salt thereof. In some embodiments, the present methods employ etifoxine, or a pharmaceutically acceptable salt thereof, as the sole active ingredient used to treat a substance abuse disorder. In some embodiments, the present methods employ etifoxine, or a pharmaceutically acceptable salt thereof, in conjunction with one or more active ingredients used to treat a substance abuse disorder. In some embodiments, etifoxine or a pharmaceutically acceptable salt thereof is administered in combination with an additional active ingredient used to treat a substance abuse disorder, e.g., co-formulated or administered separately.

Opioid use disorder includes signs and symptoms that reflect compulsive, prolonged self-administration of opioid substances that are used for no legitimate medical purpose or, if another medical condition is present that requires opioid treatment, that are used in doses greatly in excess of the amount needed for that medical condition. For example, an individual prescribed analgesic opioids for pain relief at adequate dosing will use significantly more than prescribed and not only because of persistent pain.

Substance abuse disorder, including cocaine, alcohol, and opioids has been associated with the dopamine reward pathways (Ross, S. & Peselow, E. The Neurobiology of Addictive Disorders. Clin Neuropharmacol 32, 269-276 (2009)). The neurotransmitter GABA suppresses striatal dopamine release and blunts cocaine-induced increases in extracellular dopamine in the striatum and nucleus accumbens in animals (Dewey. S. et al. GABAergic inhibition of endogenous dopamine release measured in vivo with 11C-raclopride and positron emission tomography. J Neurosci 12, 3773-3780 (1992)). Progesterone treatment has been shown to decrease craving to cocaine in clinical studies, potentially due to the GABAergic effect GABA-A positive allosteric modulator neuroactive steroids synthesized from progesterone (Sinha. R. et al. Sex steroid hormones, stress response, and drug craving in cocaine-dependent women: Implications for relapse susceptibility. Exp Clin Psychopharm 15, 445 (2007)).

Stress is an important factor in the development of substance use disorders and in perpetuating the cycle of drug use, abstinence, and relapse in addicted individuals.⁴

Progesterone treatment has been shown to decrease craving to cocaine in clinical studies, potentially due to the GABAergic effect GABA-A positive allosteric modulator neuroactive steroids synthesized from progesterone.

In some embodiments, the present disclosure provides methods of treating opioid use disorder comprising administering an effective amount of etifoxine or a pharmaceutically acceptable salt thereof. In some embodiments, the etifoxine is administered as a monotherapy. In some embodiments, the etifoxine is administered as an adjunctive to the patient's existing therapy (e.g., the current standard of care). In certain embodiments, the etifoxine is administered as an adjunctive to methadone. In certain embodiments, the etifoxine is administered as an adjunctive to buprenorphine.

In some embodiments, after said treatment the patient experiences a substantial reduction of opioid use disorder that is characterized by an abstinence to opioid use during the period of etifoxine administration. As used herein “abstinence to opioid use” means a negative urine drug test and no self-reported opioid use on the timeline follow-back (TLFB) survey during the period of etifoxine administration. TLFB survey use calendars and daily recall of substance use on specific days to record quantity or frequency of opioid use. Omission of any of these criteria resulted in failure to confirm abstinence for the week.

In some embodiments, after said treatment the patient experiences a substantial reduction of opioid use disorder that is characterized by a statistically significant decrease in the percentage of opioid-free weeks in an etifoxine treated group compared to a placebo treated group during the period of etifoxine administration (i.e., there is a significant statistical difference between the percentage of opioid-free weeks of etifoxine treatment relative to placebo treatment).

In some embodiments, after said treatment the patient experiences a substantial reduction of opioid use disorder that is characterized by substantial improvement is demonstrated by craving assessment—weekly self-report visual analogue scale (VAS) of need for opioids (scale 0-100, 0=not at all; 100=very much so). Response defined by significant statistical difference in the mean change in VAS score from baseline of treatment group relative to placebo ((Krupitsky, E. et al. Injectable extended-release naltrexone for opioid dependence: a double-blind, placebo-controlled, multicentre randomised trial. Lancet 377, 1506-1513 (2006)).

In some embodiments, after said treatment the patient experiences a substantial reduction of opioid use disorder that is characterized by a statistically significant change in retention assessment compared to a placebo treated group. As used herein, “retention assessment” means the number of days of retention on either cognitive behavioral therapy or pharmacotherapy by TLFB during the period of etifoxine administration.

In certain embodiments, after said treatment the patient experiences a statistically significant change in retention assessment that is characterized by significant statistical difference in the mean change in number of days of retention in etifoxine treatment group relative to placebo.

In some embodiments, the present disclosure provides methods of treating cocaine use disorder comprising administering an effective amount of etifoxine or a pharmaceutically acceptable salt thereof. In some embodiments, the etifoxine is administered as a monotherapy. In some embodiments, the etifoxine is administered as an adjunctive to the current standard of care. In some embodiment, the etifoxine is administered as an adjunctive to buprenorphine. In some embodiments, the etifoxine is administered as an adjunctive to buprenorphine and naloxone. In some embodiments, the etifoxine is administered as an adjunctive to naltrexone. In some embodiments, the etifoxine administered as an adjunctive to lofexidine.

In some embodiments, the present disclosure provides methods of treating alcohol use disorder comprising administering an effective amount of etifoxine or a pharmaceutically acceptable salt thereof. In some embodiments, the etifoxine is administered as a monotherapy. In some embodiments, the etifoxine is administered as an adjunctive to the current standard of care. In some embodiments, the etifoxine is administered as an adjunctive to a benzodiazepine.

In some embodiments, the present disclosure provides methods of treating benzodiazepine use disorder comprising administering an effective amount of etifoxine or a pharmaceutically acceptable salt thereof. In some embodiments, the etifoxine is administered as a monotherapy. In some embodiments, the etifoxine is administered as an adjunctive to the current standard of care. In some embodiments, the etifoxine is administered as an adjunctive to medically supervised withdrawal (detoxification). In some embodiments, the etifoxine is administered as an adjunctive to residential rehabilitation treatment. In some embodiments, the etifoxine is administered as an adjunctive to mutual help groups. In some embodiments, the etifoxine is administered as an adjunctive to outpatient substance use disorder services (e.g., counseling or medication for addiction).

Methods of Treating Dyspnea

In some embodiments, the present disclosure provides methods of treating dyspnea comprising administering an effective amount of etifoxine or a pharmaceutically acceptable salt thereof. In some embodiments, the present methods employ etifoxine, or a pharmaceutically acceptable salt thereof, as the sole active ingredient used to treat dyspnea. In some embodiments, the present methods employ etifoxine, or a pharmaceutically acceptable salt thereof, in conjunction with one or more active ingredients used to treat dyspnea. In some embodiments, etifoxine or a pharmaceutically acceptable salt thereof is administered in combination with an additional active ingredient used to treat dyspnea, e.g., co-formulated or administered separately.

Dyspnea is the clinical term used to describe the subjective feeling of impaired breathing. The term comprises a wide range of respiratory symptoms such as shortness of breath, increased respiratory rate or altered depth in respiration (American Thoracic Society. Dyspnea. Mechanisms, assessment, and management: a concensus statement. Am J Respir Crit Care Med 1999; 159:321-40.).

Asthma is a chronic inflammatory disease of the airways characterized by attacks of reversible airflow obstruction that is clinically manifested by dyspnea, wheezing, chest tightness and cough. Asthma is characterized by pathological changes in the airway smooth muscle, which lead to obstruction and bronchial hyper-reactivity.

Chronic obstructive pulmonary disease (COPD), which is defined as a progressive and irreversible limitation of functional airflow, encompasses a range of chronic respiratory diseases.

Psychiatric comorbidity is common in patients with asthma and chronic obstructive pulmonary disease (COPD) and an association between anxiety and depression and respiratory symptoms has been found in cross-sectional population studies (Dowson C, Laing R, Barraclough R, Town I, Mulder R. Norris K. et al. The use of the Hospital Anxiety and Depression Scale (HADS) in patients with chronic obstructive pulmonary disease: a pilot study. N Zealand Med J 2001; 114:447-9.).

Longitudinal studies of 515 adults followed over a 9-year period and measured for anxiety, depression, and dyspnea show a causal relationship between the onset of anxiety and depression, and dyspnea (Neuman A, Gunnbjörnsdottir M, Tunsäter A. Nyström L, Franklin K A, Norrman E, Janson C. Dyspnea in relation to symptoms of anxiety and depression: A prospective population study. Respir Med. 2006.).

Use of benzodiazepines significantly increases the risk of respiratory failure in COPD (Chen S J, Yeh C M, Chao T F, Liu C J, Wang K L, Chen T J, Chou P, Wang F D. The Use of Benzodiazepine Receptor Agonists and Risk of Respiratory Failure in Patients with Chronic Obstructive Pulmonary Disease: A Nationwide Population-Based Case-Control Study. Sleep. 2015.).

In a study of 12 patients with well controlled asthma, diazepam aerosol reduced the bronchoconstriction induced by methacholine (Miric M, Ristic S. Joksimovic B N, Medenica S, Racic M, Ristic S. Joksimovic V R, Skipina M. Reversion of methacholine induced bronchoconstriction with inhaled diazepam in patients with asthma. Rev Med Chil. 2016.). In the first day of testing, methacholine inhalation (6 mg/mL) led to a significant drop in FEV1 from 2.98 to 1.69 L. On the second day of study, in the same patients, previous inhalation with diazepam reduced the changes of FEV1 after inhalation of methacholine. This parameter decreased from 2.48 to 2.21 L.

Clinical evidence on the efficacy of benzodiazepines for the treatment of breathlessness in respiratory compromised patients is inconclusive. Meta-analysis of 8 clinical trials with a total of 240 participants comparing benzodiazepines to morphine, midazolam, or control show no statistically significant difference (Simon S T, Higginson I J, Booth S, Harding R, Weingartner V, Bausewein C. Benzodiazepines for the relief of breathlessness in advanced malignant and non-malignant diseases in adults. Cochrane Database Syst Rev. 2016.). Opioids are standard therapy for breathlessness in COPD.

Imidobenzodiazepines derivatives have shown to relax airway smooth muscle ex vivo and reduce airway hyper-responsiveness in murine asthma model (Forkuo G S, Guthrie M L, Yuan N Y, Nieman A N, Kodali R, Jahan R, Stephen M R. Yocum G T, Treven M, Poe M M, Li G, Yu O B, Hartzler B D, Zahn N M, Emst M. Emala C W, Stafford D C Cook J M, Arnold L A. Development of GABAA Receptor Subtype-Selective Imidazobenzodiazepines as Novel Asthma Treatments. Mol Pharm. 2016; Jahan R, Stephen M R, Forkuo G S, Kodali R, Guthrie M L. Nieman A N, Yuan N Y, Zahn N M, Poe M M, Li G, Yu O B, Yocum G T, Emala C W. Stafford D C, Cook J M, Arnold L A. Optimization of substituted imidazobenzodiazepines as novel asthma treatments. Eur J Med Chem. 2017.), potentially via a2b3g2 GABAa receptor subtype potentiation.

Studies suggest that GABA PAM neuroactive steroids may help with the treatment of the anxiety symptoms of dyspnea.

In some embodiments, the present disclosure provides methods of treating dyspnea comprising administering a therapeutically effective amount of etifoxine or a pharmaceutically acceptable salt thereof. In some embodiments, the etifoxine is administered as a monotherapy. In some embodiments, the etifoxine is administered as an adjunctive to the current standard of care. In some embodiments, the etifoxine is administered as an adjunctive to an opioid. In some embodiments, the opioid is selected from morphine, dihydrocodeine, and diamorphine.

In some embodiments, the present disclosure provides methods of treating dyspnea associated with respiratory conditions (asthma, chronic obstructive pulmonary disease, idiopathic pulmonary fibrosis, cancer) comprising administering a therapeutically effective amount of etifoxine or a pharmaceutically acceptable salt thereof. In some embodiments, the present disclosure provides methods of treating dyspnea associated with heart disease (such as congestive heart failure). In some embodiments, the present disclosure provides methods of treating dyspnea associated with gastroesophageal reflux disease (GERD).

In some embodiments, the present disclosure provides methods of treating dyspnea in asthma comprising administering a therapeutically effective amount of etifoxine or a pharmaceutically acceptable salt thereof.

In some embodiments, the present disclosure provides methods of treating dyspnea in COPD comprising administering a therapeutically effective amount of etifoxine or a pharmaceutically acceptable salt thereof.

In some embodiments, the present disclosure provides methods of treating dyspnea in idiopathic pulmonary fibrosis comprising administering a therapeutically effective amount of etifoxine or a pharmaceutically acceptable salt thereof.

In some embodiments, the present disclosure provides methods of treating dyspnea in lung cancer comprising administering a therapeutically effective amount of etifoxine or a pharmaceutically acceptable salt thereof.

In some embodiments, the present disclosure provides methods of treating dyspnea in other severe respiratory disorders comprising administering a therapeutically effective amount of etifoxine or a pharmaceutically acceptable salt thereof.

In one aspect, the present disclosure provides methods for treating dyspnea by administering etifoxine or a pharmaceutically acceptable salt thereof to a patient in need thereof. An effective amount is an amount sufficient to eliminate or significantly reduce dyspnea symptoms or to alleviate those symptoms (e.g., reduce the symptoms, such as breathlessness or affective distress (e.g., measurement of how distressing breathing feels to the patient), compared to the symptoms present prior to treatment).

Reduction of dyspnea in patients (including COPD or IPF patients) can be determined by various methods. In some embodiments, the effectiveness of a dosage regimen can be determined by evaluation via an EXACT-Respiratory Symptoms (E-RSTM) breathlessness subscale score, Borg dyspnea scale value total score, Borg dyspnea scale domains (sensory-perceptual, affective distress or symptom impact), numerical rating scale dyspnea value, Modified Medical Research Council Scale, PROMIS Pool v1.0 Dyspnea Emotional Response Scale, PROMIS Item Bank v1.0 Dyspnea Severity-Short Form 10a Scale, PROMIS Item Bank v1.0 Dyspnea Characteristics Scale or any combination thereof.

In some embodiments, after said treatment the patient experiences a reduction of dyspnea that is characterized by at least a 1.0 point reduction in the EXACT-Respiratory Symptoms (E-RSTM) breathlessness subscale score compared to prior to the treatment. In some embodiments, the reduction of dyspnea is characterized by a decline in EXACT-Respiratory Symptoms (E-RSTM) breathlessness subscale score ranging from about 0.0 to about 23.0 points (Bacci E D, O'Quinn S, Leidy N K, Murray L, Vernon M. Evaluation of a respiratory symptom diary for clinical studies of idiopathic pulmonary fibrosis. Respir Med. 2018 Januray; 134:130-138.), for example, about 1.0 point, about 3.0 points, about 5.0 points, about 7.0 points, about 9.0 points, about 11.0 points, about 13.0 points, about 15.0 points, about 17.0 points, about 19 points, about 21 points and about 23.0 points compared to prior to the treatment.

In some embodiments, after said treatment the patient experiences a reduction of dyspnea that is characterized by at least a one point change in the Borg dyspnea scale value total score compared to prior to the treatment. In some embodiments, the reduction of dyspnea is characterized by a decline in Borg dyspnea scale value total score ranging from about 0.5 to about 2.0 points, for example, about 0.5 points, about 1.0 point, about 1.5 points and about 2.0 points compared to prior to the treatment.

In some embodiments, after said treatment the patient experiences a reduction of dyspnea that is characterized by at least a one point change in any of the Borg dyspnea scale domains (sensory-perceptual, affective distress or symptom impact) compared to prior to the treatment. In some embodiments, the reduction of dyspnea is characterized by a decline in Borg dyspnea scale domains (sensory-perceptual, affective distress or symptom impact) ranging from about 0.5 to about 2.0 points, for example, about 0.5 points, about 1.0 point, about 1.5 points and about 2.0 points compared to prior to the treatment.

In some embodiments, after said treatment the patient experiences a reduction of dyspnea that is characterized by at least a 3 point reduction in the numerical rating scale dyspnea value compared to prior to the treatment. In some embodiments, the reduction of dyspnea is characterized by a decline in Borg dyspnea scale domains (sensory-perceptual, affective distress or symptom impact) ranging from about 2.0 to about 5.0 points, for example, about 2.0 points, about 2.5 points, about 3.0 point, about 3.5 points, about 4.0 points, about 4.5 points and about 5.0 points compared to prior to the treatment

In some embodiments, after said treatment the patient experiences a reduction of dyspnea that is characterized by at least a one category change in the Modified Medical Research Council Scale compared to prior to the treatment. In some embodiments, the reduction of dyspnea is characterized by an improvement in Modified Medical Research Council Scale ranging from at least one category to about three categories, for example, about one category, about two categories, and about three categories compared to prior to the treatment.

In some embodiments, after said treatment the patient experiences a reduction of dyspnea that is characterized by at least a one category change in at least one of the 7 questions of the PROMIS Pool v1.0 Dyspnea Emotional Response Scale compared to prior to the treatment. In some embodiments, the reduction of dyspnea is characterized by an improvement in at least one of the 7 questions of the PROMIS Pool v1.0 Dyspnea Emotional Response Scale ranging from at least one category to about three categories, for example, about one category, about two categories, and about three categories compared to prior to the treatment.

In some embodiments, after said treatment the patient experiences a reduction of dyspnea that is characterized by at least a one category change in at least one of the 10 questions of the PROMIS Item Bank v1.0 Dyspnea Severity-Short Form 10a Scale compared to prior to the treatment. In some embodiments, the reduction of dyspnea is characterized by an improvement in at least one of the 10 questions of the PROMIS Item Bank v1.0 Dyspnea Severity-Short Form 10a Scale ranging from at least one category to about three categories, for example, about one category, about two categories, and about three categories compared to prior to the treatment.

In some embodiments, after said treatment the patient experiences a reduction of dyspnea that is characterized by at least a one category change in at least one of the 4 items or a one category change in the question “I have been short of breath” of the PROMIS Item Bank v1.0 Dyspnea Characteristics Scale compared to prior to the treatment. In some embodiments, the reduction of dyspnea is characterized by an improvement in at least one of the 4 items and/or the question “I have been short of breath” of the PROMIS Item Bank v1.0 Dyspnea Characteristics Scale ranging from at least one category to about three categories, for example, about one category, about two categories, and about three categories compared to prior to the treatment.

Reduction of dyspnea in patients (including COPD or IPF patients) can be determined by a reduction of the patient's affective distress (i.e., measurement of how distressing breathing feels to the patient). In some embodiments, the patient's affective distress can be determined using a multi-item anxiety scale. Reduction of anxiety in patients can be determined by various methods.

The Hamilton Rating Scale for Anxiety (HAM-A) is an anxiety rating scale consisting of 14 items that assess anxious mood, tension, fear, insomnia, intellectual (cognitive) functioning, depressed mood, behavior at interview, somatic (sensory), cardiovascular, respiratory, gastrointestinal, genitourinary, autonomic, and somatic (muscular) symptom (Hamilton. M. The Assessment of Anxiety States by Rating, Br J Med Psychol. (1959):32 (1), pages 50-55.). Each symptom is rated from 0 (absent) to 4 (maximum severity) scale. The total score is used to categorize the severity of anxiety: mild severity (total score less than 17), mild to moderate severity (total score between 18-24), and moderate to severe (total score between 25-30). Total scores range from 0 to 56 with higher scores indicating greater severity.

In some embodiments, after the treatment the patient experiences a substantial reduction of dyspnea that is characterized by at least about a 30% decline in total HAM-A value compared to prior to the treatment. In some embodiments, the reduction of dyspnea is characterized by a decline in HAM-A value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of dyspnea that is characterized by at least a one point decline in HAM-A value compared to prior to the treatment. In some embodiments, the reduction of dyspnea is characterized by a decline in HAM-A value ranging from about one point to about five points, for example, about one point, about two points, about three points, about four points, and about five points compared to prior to the treatment. In some embodiments, the reduction of dyspnea is characterized by a decline HAM-A value of about two points. In some embodiments, the reduction of dyspnea is characterized by a decline in HAM-A value of about three points. In some embodiments, the reduction of dyspnea is characterized by a decline in HAM-A value of about four points. In some embodiments, the reduction of depression is characterized by a decline in HAM-A value of about five points.

In some embodiments, after the treatment the patient experiences a substantial reduction of dyspnea that is characterized by at least a one category change in HAM-A severity classification compared to prior to the treatment. In some embodiments, the reduction of dyspnea is characterized by a one category change HAM-A severity classification compared to prior to the treatment. In some embodiments, the reduction of dyspnea is characterized by a two category change HAM-A severity classification compared to prior to the treatment. In some embodiments, the reduction of depression is characterized by a three category change HAM-A severity classification compared to prior to the treatment.

The General Anxiety Disorder 7 (GAD-7) is an anxiety rating scale consisting of 7 (Spitzer R. L. et al., A brief measure for assessing generalized anxiety disorder. Arch Intern Med. 2006; 166: 1092-1097.). Each symptom is rated from 0 (Not at all Sure) to 4 (Nearly Every Day) scale. Total scores range from 0 to 21 with higher scores indicating greater severity. The total score is used to categorize the severity of anxiety: minimal anxiety (total score between 0-4), mild anxiety (total score between 5-9), moderate anxiety (total score between 10-14) and severe anxiety (total score between 15-21).

In some embodiments, after the treatment the patient experiences a substantial reduction of dyspnea that is characterized by at least about a 20% decline in total GAD-7 value compared to prior to the treatment. In some embodiments, the reduction of dyspnea is characterized by a decline in GAD-7 value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of dyspnea that is characterized by at least a one point decline in GAD-7 value compared to prior to the treatment. In some embodiments, the reduction of dyspnea is characterized by a decline in GAD-7 value ranging from about one point to about ten points, for example, about one point, about two points, about three points, about four points, about five points, about six points, about seven points, about eight points, about nine points and about ten points compared to prior to the treatment. In some embodiments, the reduction of dyspnea is characterized by a decline GAD-7 value of about two points. In some embodiments, the reduction of dyspnea is characterized by a decline in GAD-7 value of about three points. In some embodiments, the reduction of dyspnea is characterized by a decline in GAD-7 value of about four points. In some embodiments, the reduction of dyspnea is characterized by a decline in GAD-7 value of about five points. In some embodiments, the reduction of dyspnea is characterized by a decline GAD-7 value of about six points. In some embodiments, the reduction of dyspnea is characterized by a decline in GAD-7 value of about seven points. In some embodiments, the reduction of dyspnea is characterized by a decline in GAD-7 value of about eight points. In some embodiments, the reduction of dyspnea is characterized by a decline in GAD-7 value of about nine points. In some embodiments, the reduction of dyspnea is characterized by a decline in GAD-7 value of about ten points.

In some embodiments, after the treatment the patient experiences a substantial reduction of dyspnea that is characterized by at least a one category change in GAD-7 severity classification compared to prior to the treatment. In some embodiments, the reduction of dyspnea is characterized by a one category change GAD-7 severity classification compared to prior to the treatment. In some embodiments, the reduction of dyspnea is characterized by a two category change GAD-7 severity classification compared to prior to the treatment. In some embodiments, the reduction of dyspnea is characterized by a three category change GAD-7 severity classification compared to prior to the treatment.

Methods of Treating Tinnitus

Tinnitus is the perception of sound without an external source. Tinnitus is common with some studies indicating that more than 50 million people in the United States report tinnitus (J. A. Henry, et al., General review of tinnitus: prevalence, mechanisms, effects, and management. J Speech Lang Hear res. 2005, 48, 1204-1235.). Tinnitus is a symptom, rather than a disease, that can be associated with multiple causes and aggravating co-factors. In rare cases, tinnitus is a symptom of serious, potentially life-threatening disease (for example, vascular tumor or vestibular schwannoma (VS)).

Tinnitus can be persistent, bothersome, and costly. A 1994 U.S. National Health Interview Survey asked whether individuals experienced “ringing, roaring, or buzzing in the ears that lasted for at least 3 months.” Such tinnitus was present in 1.6% of adults ages 18 to 44 years, 4.6% of adults ages 45 to 64 years, and 9.0% of adults 60 years and older (Vital and Health Statistics: Current Estimates from the National Health Interview Survey, 1994. Series 10: Data from the National Health Survey No. 193; US Department of Health and Human Services Public Health Service, CDC, National Center for Health Statistics, DHHS Publication No. (PHS) 96-1521.). In another study of more than 3000 adults between the ages of 21 and 84 years, 10.6% of respondents reported tinnitus of at least moderate severity or causing difficulty falling asleep (Nondahl D M, Cruickshanks K J, Huang G H, et al. Tinnitus and its risk factors in the Beaver Dam Offspring Study. Int J Audiol. 2011; 50(5):313-320.). Tinnitus may have large economic consequences. For example, tinnitus was the most prevalent service-connected disability for U.S. military veterans receiving compensation at the end of fiscal year 2012, resulting in nearly 1 million veterans receiving disability awards (US Department of Veterans Affairs, ed. Annual Benefits Report: Fiscal Year 2012. Washington, D.C.: Department of Veterans Affairs; 2013.).

Tinnitus can occur on one or both sides of the head and can be perceived as coming from within or outside the head. Tinnitus most often occurs in the setting of concomitant sensorineural hearing loss (SNHL), particularly among patients with bothersome tinnitus and no obvious ear pathology. The quality of tinnitus can also vary, with ringing, buzzing, clicking, pulsations, roaring and other noises described by tinnitus patients. In addition, the effects of tinnitus on health-related quality of life (QOL) vary widely, with most patients less severely affected but some experiencing anxiety, depression, and extreme life changes. Patients who have tinnitus accompanied by severe anxiety or depression require prompt identification and intervention, as suicide has been reported in tinnitus patients who have coexisting psychiatric illness.

Tinnitus may be categorized as (1) subjective or objective, (2) primary or secondary, (3) acute or chronic and (4) on the basis of its severity. Most tinnitus is subjective, i.e., perceived only by the patient. In contrast, objective tinnitus, which is rare, can be perceived by others, for example, by a physician who can hear the tinnitus or detect the nerve signals causing it.

Primary tinnitus is tinnitus that is idiopathic and may or may not be associated with sensorineural hearing loss. Currently, there is no cure for primary tinnitus and therapies are directed to providing symptomatic relief, including education and counseling, auditory therapies such as hearing aids and specific forms of sound therapy, cognitive behavioral therapy (CBT), dietary changes and supplements, acupuncture, and transcranial magnetic stimulation (TMS).

Secondary tinnitus is tinnitus that is associated with a specific underlying cause (other than SNHL) or an identifiable organic condition. Secondary tinnitus is a symptom of a range of auditory and non-auditory system disorders that include simple cerumen impaction of the external auditory canal, middle ear diseases such as otosclerosis or Eustachian tube dysfunction, cochlear abnormalities such as Meniere's disease, and auditory nerve pathology such as VS. Non-auditory system disorders that can cause tinnitus include vascular anomalies, myoclonus, and intracranial hypertension. Management of secondary tinnitus is generally targeted toward identification and treatment of the specific underlying condition.

Acute tinnitus is tinnitus that lasts less than 6 months while chronic tinnitus lasts 6 months or longer. In some patients, tinnitus severity is mild and hardly affects day-to-day activities. In more severe tinnitus, sounds are constantly and clearly heard, which can negatively and severely impact daily life and work by, for example, making it difficult for the patient to sleep and concentrate properly. Despite its prevalence and debilitating effects, there are no FDA-approved medicines for the treatment of tinnitus.

Etifoxine is a TSPO agonist that increases neuroactive steroid synthesis and increases levels of allopregnanolone and other positive endogenously synthesized GABA-A positive allosteric modulator neuroactive steroids. Hence, etifoxine indirectly enhances GABA-A neurotransmission by increase of endogenous neuroactive steroid levels.

Non-clinical models of tinnitus demonstrate that the emergence of tinnitus is associated with hyperactivity of the dorsal cochlear nucleus due to decrease in GABAergic inhibition (Middleton, J. W. et al. Mice with behavioral evidence of tinnitus exhibit dorsal cochlear nucleus hyperactivity because of decreased GABAergic inhibition. Proc National Acad Sci 108, 7601-7606 (2011)) and decrease of inhibitory synaptic transmission (Brozoski, T. J., Spires, J. T. & Bauer, C. A. Vigabatrin, a GABA Transaminase Inhibitor. Reversibly Eliminates Tinnitus in an Animal Model. J Assoc Res Otolaryngology 8, 105-118 (2007)). Non-clinical models of tinnitus show that drugs that increase GABAergic inhibition (i.e., vigabatrin), but not those that reduce excitation (i.e., ketamine) reverse the tinnitus behavior (Shulman, A., Rashun, A. & Goldstein, B. A. GABAA-benzodiazepine-chloride receptor-targeted therapy for tinnitus control: preliminary report. Int Tinnitus J 8, 30-6 (2002)). Clinical studies with GABA-A receptor modulators, such as clonazepam, are reported to control some symptoms of tinnitus (Yang, S., Weiner, B. D., Zhang, L. S., Cho, S.-J. & Bao. S. Homeostatic plasticity drives tinnitus perception in an animal model. Proc National Acad Sci 108, 14974-14979 (2011)).

In some embodiments, the present disclosure provides methods of treating tinnitus by administering a therapeutically effective amount of etifoxine or a pharmaceutically acceptable salt thereof. In some embodiments, etifoxine or a pharmaceutically acceptable salt thereof is administered as the sole active ingredient. In some embodiments, etifoxine or a pharmaceutically acceptable salt thereof is administered in combination with repetitive transcranial magnetic stimulation (rTMS) for the treatment of tinnitus.

Reduction of tinnitus in patients with tinnitus-causing conditions can be determined by various methods. In some embodiments, the effectiveness of a dosage regimen can be determined by evaluation via Tinnitus Handicap Inventory (THI) value. In some embodiments, the effectiveness of a dosage regimen can be determined by evaluation via a total Tinnitus Handicap Inventory (THQ) value. In some embodiments, the effectiveness of a dosage regimen can be determined by evaluation via total Tinnitus Reaction Questionnaire (TRQ) value. In some embodiments, the effectiveness of a dosage regimen can be determined by evaluation via total Tinnitus Functional Index (TFI) value. In yet some other embodiments, the effectiveness of a dosage regimen can be determined by evaluation via total THI value, THQ value, total TRQ value, total TFI value, or any combination thereof.

According to some embodiments of the present disclosure, the dosing frequency and dose amount per administration of etifoxine are selected to provide therapeutic effects for the treatment of acute peripheral tinnitus.

According to some embodiments of the present disclosure, the dosing frequency and dose amount per administration of etifoxine are selected to provide therapeutic effects for the treatment of chronic tinnitus.

According to some embodiments of the present disclosure, the dosing frequency and dose amount per administration of etifoxine are selected to provide therapeutic effects for the treatment of chronic subjective tinnitus.

According to some embodiments of the present disclosure, the dosing frequency and dose amount per administration of etifoxine are selected to provide therapeutic effects for the treatment of drug-induced tinnitus (e.g., caused by administration of ototoxic drugs). In certain embodiments, the drug that induces tinnitus is selected from the group consisting of cisplatin, ACE inhibitors, amlodipine, nicardipine, atorvastatin, azithromycin, mechlorethamine, vincristine, benzodiazepines, ciprofloxacin, gentamicin, tobramycin, bumetanide, ethacrynic acid, furosemide, isotretinoin, amitriptyline, nortriptyline, and quinine medications.

According to some embodiments of the present disclosure, the dosing frequency and dose amount per administration of etifoxine are selected to provide therapeutic effects for the treatment of tinnitus caused by an otologic condition. In some embodiments, the otologic condition is selected from the group consisting of hearing loss (including noise-induced hearing loss), presbycusis, otosclerosis, otitis, impacted cerumen, sudden deafness, and Meniere's disease.

According to some embodiments of the present disclosure, the dosing frequency and dose amount per administration of etifoxine are selected to provide therapeutic effects for the treatment of tinnitus caused by vascular disease or disorder. In some embodiments, the vascular disease or disorder is selected from the group consisting of carotid atherosclerosis, arteriovenous malformation, and hypertension.

According to some embodiments of the present disclosure, the dosing frequency and dose amount per administration of etifoxine are selected to provide therapeutic effects for the treatment of tinnitus caused by a neurologic condition. In some embodiments, the neurologic condition is selected from the group consisting of head or neck injury, whiplash, multiple sclerosis, vestibular schwannoma (commonly called an acoustic neuroma), and cerebellopontine angle tumor.

According to some embodiments of the present disclosure, the dosing frequency and dose amount per administration of etifoxine are selected to provide therapeutic effects for the treatment of tinnitus caused by an infectious condition. In some embodiments, the infectious condition is selected from the group consisting of otitis media and sequelae of Lyme disease, meningitis, and syphilis.

According to some embodiments, the substantial reduction in tinnitus provided by the methods of the present disclosure requires treatment for a specified time interval (e.g., at least one week) before the patient experiences substantial reduction of tinnitus (i.e., there is an induction period before the patient experiences a substantial reduction in tinnitus). In some embodiments, after treatment for at least one day, at least two days, at least three days, at least four days, at least five days, at least six days, at least seven days or at least eight days, the patient experiences a substantial reduction of tinnitus compared to prior to the treatment.

In some embodiments, after treatment for at least one week, at least two weeks, at least three weeks, at least four weeks, at least five weeks, at least six weeks, at least seven weeks or at least eight weeks, the patient experiences a substantial reduction of tinnitus compared to prior to the treatment. In certain embodiments, after treatment for at least one week the patient experiences a substantial reduction of tinnitus compared to prior to the treatment. According to this embodiment, the substantial reduction in tinnitus may be expressed using any of the methods described herein (for example, decline in total Tinnitus Handicap Questionnaire (THQ) value compared to prior to the treatment, improvement in the total Tinnitus Handicap Inventory (THI) value compared to prior to the treatment, etc.).

The Tinnitus Handicap Inventory (THI) (Brozoski, T. J., Spires, J. T. & Bauer, C. A. Vigabatrin, a GABA Transaminase Inhibitor, Reversibly Eliminates Tinnitus in an Animal Model. J Assoc Res Otolaryngology 8, 105-118 (2007); Middleton, J. W. et al. Mice with behavioral evidence of tinnitus exhibit dorsal cochlear nucleus hyperactivity because of decreased GABAergic inhibition. Proc National Acad Sci 108, 7601-7606 (2011); Newman C W, et al., Development of the Tinnitus Handicap Inventory. Archives of Otolaryngology—Head and Neck Surgery, 1996, 122(2):143-8; McCombe, A., et al., Guidelines for the Grading of Tinnitus Severity: the Results of a Working Group Commissioned by the British Association of Otolaryngologists, Head and Neck Surgeons, Clinical Otolarynogology, 2001, 26, 388-393.) is a tinnitus rating scale consisting of 25 items scored on a 3-category rating scale (yes: no and maybe) where each category is assigned a point value (yes=4 points: sometimes=2 points; no=0 points) with a possible total score of 100 points. There are five severity categories in the THI scale: Grade 1, slight or no handicap (0-16 points); Grade 2, mild handicap (18-36 points): Grade 3, moderate handicap (38-56 points); Grade 4, severe handicap (58-76 points); and Grade 5, catastrophic handicap (78-100 points). The higher the score, the greater the tinnitus symptoms. Therefore, a decrease in the total score or on individual item scores indicates improvement.

In some embodiments, after the treatment the patient experiences a substantial reduction of tinnitus that is characterized by at least about a 10% decline in total Tinnitus Handicap Inventory (THI) value compared to prior to the treatment. In some embodiments, the reduction of tinnitus is characterized by a decline in THI value ranging from about 10% to about 100%, for example, about 10%, about 20%, about 30%, about 40%, about 50%, about 60%, about 70%, about 80%, about 90%, and about 100%, compared to prior to the treatment.

In some embodiments, after the treatment the patient experiences a substantial reduction of tinnitus that is characterized by at least a two-point decline in THI value compared to prior to the treatment. In some embodiments, the reduction of tinnitus is characterized by a decline in THI value ranging from about two point to about eighty points, for example, about two points, about four points, about six points, about eight points, about ten points, about twelve points, about fourteen points, about sixteen points, about eighteen points, about twenty points, about twenty-two points, about twenty-four points, about twenty-six points, about twenty-eight points, about thirty points, about thirty-two points, about thirty-four points, about thirty-six points, about thirty-eight points, about forty points, about forty-two points, about forty-four points, about forty-six points, about forty-eight points, about fifty points, about fifty-two points, about fifty-four points, about fifty-six points, about fifty-eight points, about sixty points, about sixty-two points, about sixty-four points, about sixty-six points, about sixty-eight points, about seventy points, about seventy-two points, about seventy-four points, about seventy-six points, about seventy-eight points, or about eighty points.

In some embodiments, the reduction of tinnitus is characterized by a decline in THI value of about two points. In some embodiments, the reduction of tinnitus is characterized by a decline in THI value of about four points. In some embodiments, the reduction of tinnitus is characterized by a decline TI value of about six points. In some embodiments, the reduction of tinnitus is characterized by a decline in THI value of about eight points. In some embodiments, the reduction of tinnitus is characterized by a decline THI value of about ten points. In some embodiments, the reduction of tinnitus is characterized by a decline in THI value of about twelve points. In some embodiments, the reduction of tinnitus is characterized by a decline THI value of about fourteen points. In some embodiments, the reduction of tinnitus is characterized by a decline in THI value of about sixteen points. In some embodiments, the reduction of tinnitus is characterized by a decline THI value of about eighteen points. In some embodiments, the reduction of tinnitus is characterized by a decline in THI value of about twenty points. In some embodiments, the reduction of tinnitus is characterized by a decline THI value of about twenty-four points. In some embodiments, the reduction of tinnitus is characterized by a decline TI value of about twenty-six points. In some embodiments, the reduction of tinnitus is characterized by a decline THI value of about twenty-eight points. In some embodiments, the reduction of tinnitus is characterized by a decline TI value of about thirty points. In some embodiments, the reduction of tinnitus is characterized by a decline THI value of about thirty-two points. In some embodiments, the reduction of tinnitus is characterized by a decline THI value of about thirty-four points. In some embodiments, the reduction of tinnitus is characterized by a decline THI value of about thirty-six points. In some embodiments, the reduction of tinnitus is characterized by a decline THI value of about thirty-eight points. In some embodiments, the reduction of tinnitus is characterized by a decline THI value of about forty points. In some embodiments, the reduction of tinnitus is characterized by a decline THI value of about forty-two points. In some embodiments, the reduction of tinnitus is characterized by a decline THI value of about forty-four points. In some embodiments, the reduction of tinnitus is characterized by a decline THI value of about forty-six points. In some embodiments, the reduction of tinnitus is characterized by a decline THI value of about forty-eight points. In some embodiments, the reduction of tinnitus is characterized by a decline THI value of about fifty points. In some embodiments, the reduction of tinnitus is characterized by a decline THI value of about fifty-two points. In some embodiments, the reduction of tinnitus is characterized by a decline THI value of about fifty-four points. In some embodiments, the reduction of tinnitus is characterized by a decline THI value of about fifty-six points. In some embodiments, the reduction of tinnitus is characterized by a decline THI value of about fifty-eight points. In some embodiments, the reduction of tinnitus is characterized by a decline TI value of about sixty points. In some embodiments, the reduction of tinnitus is characterized by a decline THI value of about sixty-two points. In some embodiments, the reduction of tinnitus is characterized by a decline THI value of about sixty-four points. In some embodiments, the reduction of tinnitus is characterized by a decline THI value of about sixty-six points. In some embodiments, the reduction of tinnitus is characterized by a decline THI value of about sixty-eight points. In some embodiments, the reduction of tinnitus is characterized by a decline THI value of about seventy points. In some embodiments, the reduction of tinnitus is characterized by a decline THI value of about seventy-two points. In some embodiments, the reduction of tinnitus is characterized by a decline THI value of about seventy-four points. In some embodiments, the reduction of tinnitus is characterized by a decline THI value of about seventy-six points. In some embodiments, the reduction of tinnitus is characterized by a decline THI value of about seventy-eight points. In some embodiments, the reduction of tinnitus is characterized by a decline THI value of about eighty points.

In some embodiments, after the treatment the patient experiences a substantial reduction of tinnitus that is characterized by at least a one-category change in THI severity classification compared to prior to the treatment. In some embodiments, the reduction of tinnitus is characterized by a one-category change THI severity classification compared to prior to the treatment. In some embodiments, the reduction of tinnitus is characterized by a two-category change THI severity classification compared to prior to the treatment. In some embodiments, the reduction of tinnitus is characterized by a three-category change THI severity classification compared to prior to the treatment. In some embodiments, the reduction of tinnitus is characterized by a four-category change THI severity classification compared to prior to the treatment.

The Tinnitus Handicap Questionnaire (THQ) is a tinnitus rating scale consisting of 27 items where for each item the patient provides a numeric value from 0 (strongly disagrees) to 100 (strongly agrees) (Kuk F K, et al., The psychometric properties of a tinnitus handicap questionnaire. Ear Hear, 1990, 11(6): 434-442.). The THQ is divided into three factors: Factor 1—Social, Emotional, and Behavioral Tinnitus Effects, Factor 2—Tinnitus and Hearing, and Factor 3—Outlook on tinnitus. The score for each Factor is determined by (the highest score for each Factor is 100): Factor 1 score: (Add responses to items 4, 7, 9, 11, 13, 14, 15, 16, 17, 18, 19, 23, 24, 25, and 27)/15: Factor 2 score: (Add responses to items 2, 6, 10, 12, 20, 21, 22, and 26)=/8; Factor 3 score: (Add responses to items 3 and 5+[100−response to item 8]+[100−response to item 1])=/4.

The total THQ value is determined by [(Factor 1 score×15/27)+(Factor 2 score×8/27)+(Factor 3 score×4/27)]. The highest total THQ value is 100. The higher the score, the greater the tinnitus symptoms. Therefore, a decrease in the total score or on individual item scores or on factor scores indicates improvement.

In some embodiments, after the treatment the patient experiences a substantial reduction of tinnitus that is characterized by at least about a 10% decline in total Tinnitus Handicap Questionnaire (THQ) value, THQ Factor 1 value, THQ Factor 2 value, or THQ Factor 3 value compared to prior to the treatment. In some embodiments, the reduction of tinnitus is characterized by a decline in total THQ value, THQ Factor 1 value, THQ Factor 2 value, or THQ Factor 3 value ranging from about 10% to about 100%, for example, about 10%, about 20%, about 30%, about 40%, about 50%, about 60%, about 70%, about 80%, about 90%, and about 100%, compared to prior to the treatment.

In some embodiments, after the treatment the patient experiences a substantial reduction of tinnitus that is characterized by at least a two point decline in total THQ value, THQ Factor 1 value, THQ Factor 2 value or THQ Factor 3 value compared to prior to the treatment. In some embodiments, the reduction of tinnitus is characterized by a decline in total THQ value, THQ Factor 1 value, THQ Factor 2 value or THQ Factor 3 value ranging from about two point to about eighty points, for example, about two points, about four points, about six points, about eight points, about ten points, about twelve points, about fourteen points, about sixteen points, about eighteen points, about twenty points, about twenty-two points, about twenty-four points, about twenty-six points, about twenty-eight points, about thirty points, about thirty-two points, about thirty-four points, about thirty-six points, about thirty-eight points, about forty points, about forty-two points, about forty-four points, about forty-six points, about forty-eight points, about fifty points, about fifty-two points, about fifty-four points, about fifty-six points, about fifty-eight points, about sixty points, about sixty-two points, about sixty-four points, about sixty-six points, about sixty-eight points, about seventy points, about seventy-two points, about seventy-four points, about seventy-six points, about seventy-eight points, or about eighty points.

In some embodiments, the reduction of tinnitus is characterized by a decline in total THQ value, THQ Factor 1 value, THQ Factor 2 value or THQ Factor 3 value of about two points. In some embodiments, the reduction of tinnitus is characterized by a decline in total THQ value, THQ Factor 1 value, THQ Factor 2 value or THQ Factor 3 value of about four points. In some embodiments, the reduction of tinnitus is characterized by a decline total THQ value, THQ Factor 1 value, THQ Factor 2 value or THQ Factor 3 value of about six points. In some embodiments, the reduction of tinnitus is characterized by a decline in total THQ value, THQ Factor 1 value, THQ Factor 2 value or THQ Factor 3 value of about eight points. In some embodiments, the reduction of tinnitus is characterized by a decline total THQ value, THQ Factor 1 value, THQ Factor 2 value or THQ Factor 3 value of about ten points. In some embodiments, the reduction of tinnitus is characterized by a decline in total THQ value, THQ Factor 1 value, THQ Factor 2 value or THQ Factor 3 value of about twelve points. In some embodiments, the reduction of tinnitus is characterized by a decline total THQ value, THQ Factor 1 value, THQ Factor 2 value or THQ Factor 3 value of about fourteen points. In some embodiments, the reduction of tinnitus is characterized by a decline in total THQ value, THQ Factor 1 value, THQ Factor 2 value or THQ Factor 3 value of about sixteen points. In some embodiments, the reduction of tinnitus is characterized by a decline total THQ value, THQ Factor 1 value, THQ Factor 2 value or THQ Factor 3 value of about eighteen points. In some embodiments, the reduction of tinnitus is characterized by a decline in total THQ value, THQ Factor 1 value, THQ Factor 2 value or THQ Factor 3 value of about twenty points. In some embodiments, the reduction of tinnitus is characterized by a decline in total THQ value, THQ Factor 1 value, THQ Factor 2 value or THQ Factor 3 value of about twenty-two points. In some embodiments, the reduction of tinnitus is characterized by a decline in total THQ value, THQ Factor 1 value, THQ Factor 2 value or THQ Factor 3 value of about twenty-four points. In some embodiments, the reduction of tinnitus is characterized by a decline in total THQ value, THQ Factor 1 value, THQ Factor 2 value or THQ Factor 3 value of about twenty-six points. In some embodiments, the reduction of tinnitus is characterized by a decline in total THQ value, THQ Factor 1 value, THQ Factor 2 value or THQ Factor 3 value of about twenty-eight points. In some embodiments, the reduction of tinnitus is characterized by a decline in total THQ value, THQ Factor 1 value, THQ Factor 2 value or THQ Factor 3 value of about thirty points. In some embodiments, the reduction of tinnitus is characterized by a decline in total THQ value, THQ Factor 1 value, THQ Factor 2 value or THQ Factor 3 value of about thirty-two points. In some embodiments, the reduction of tinnitus is characterized by a decline in total THQ value, THQ Factor 1 value, THQ Factor 2 value or THQ Factor 3 value of about thirty-four points. In some embodiments, the reduction of tinnitus is characterized by a decline in total THQ value, THQ Factor 1 value, THQ Factor 2 value or THQ Factor 3 value of about thirty-six points. In some embodiments, the reduction of tinnitus is characterized by a decline in total THQ value, THQ Factor 1 value, THQ Factor 2 value or THQ Factor 3 value of about thirty-eight points. In some embodiments, the reduction of tinnitus is characterized by a decline in total THQ value, THQ Factor 1 value, THQ Factor 2 value or THQ Factor 3 value of about forty points. In some embodiments, the reduction of tinnitus is characterized by a decline in total THQ value, THQ Factor 1 value, THQ Factor 2 value or THQ Factor 3 value of about forty-two points. In some embodiments, the reduction of tinnitus is characterized by a decline in total THQ value, THQ Factor 1 value, THQ Factor 2 value or THQ Factor 3 value of about forty-four points. In some embodiments, the reduction of tinnitus is characterized by a decline in total THQ value, THQ Factor 1 value, THQ Factor 2 value or THQ Factor 3 value of about forty-six points. In some embodiments, the reduction of tinnitus is characterized by a decline in total THQ value, THQ Factor 1 value, THQ Factor 2 value or THQ Factor 3 value of about forty-eight points. In some embodiments, the reduction of tinnitus is characterized by a decline in total THQ value, THQ Factor 1 value, THQ Factor 2 value or THQ Factor 3 value of about fifty points. In some embodiments, the reduction of tinnitus is characterized by a decline in total THQ value, THQ Factor 1 value, THQ Factor 2 value or THQ Factor 3 value of about fifty-two points. In some embodiments, the reduction of tinnitus is characterized by a decline in total THQ value, THQ Factor 1 value, THQ Factor 2 value or THQ Factor 3 value of about fifty-four points. In some embodiments, the reduction of tinnitus is characterized by a decline in total THQ value, THQ Factor 1 value, THQ Factor 2 value or THQ Factor 3 value of about fifty-six points. In some embodiments, the reduction of tinnitus is characterized by a decline in total THQ value, THQ Factor 1 value, THQ Factor 2 value or THQ Factor 3 value of about fifty-eight points. In some embodiments, the reduction of tinnitus is characterized by a decline in total THQ value, THQ Factor 1 value, THQ Factor 2 value or THQ Factor 3 value of about sixty points. In some embodiments, the reduction of tinnitus is characterized by a decline in total THQ value, THQ Factor 1 value. THQ Factor 2 value or THQ Factor 3 value of about sixty-two points. In some embodiments, the reduction of tinnitus is characterized by a decline in total THQ value, THQ Factor 1 value, THQ Factor 2 value or THQ Factor 3 value of about sixty-four points. In some embodiments, the reduction of tinnitus is characterized by a decline in total THQ value, THQ Factor 1 value, THQ Factor 2 value or THQ Factor 3 value of about sixty-six points. In some embodiments, the reduction of tinnitus is characterized by a decline in total THQ value, THQ Factor 1 value, THQ Factor 2 value or THQ Factor 3 value of about sixty-eight points. In some embodiments, the reduction of tinnitus is characterized by a decline in total THQ value, THQ Factor 1 value, THQ Factor 2 value or THQ Factor 3 value of about seventy points. In some embodiments, the reduction of tinnitus is characterized by a decline in total THQ value, THQ Factor 1 value, THQ Factor 2 value or THQ Factor 3 value of about seventy-two points. In some embodiments, the reduction of tinnitus is characterized by a decline in total THQ value, THQ Factor 1 value, THQ Factor 2 value or THQ Factor 3 value of about seventy-four points. In some embodiments, the reduction of tinnitus is characterized by a decline in total THQ value, THQ Factor 1 value, THQ Factor 2 value or THQ Factor 3 value of about seventy-six points. In some embodiments, the reduction of tinnitus is characterized by a decline in total THQ value, THQ Factor 1 value, THQ Factor 2 value or THQ Factor 3 value of about seventy-eight points. In some embodiments, the reduction of tinnitus is characterized by a decline in total THQ value, THQ Factor 1 value, THQ Factor 2 value or THQ Factor 3 value of about eighty points.

The Tinnitus Reaction Questionnaire (TRQ) is a tinnitus rating scale consisting of 26 items scored on a 5-category rating scale (Not at all; A little of the time; Some of the time; A good deal of the time; Almost all of the time) where each category is assigned a point value (Not at all=0; A little of the time=1; Some of the time=2; A good deal of the time=3; Almost all of the time=4) with a possible total score of 104 points. There are five severity categories in the TRQ scale: Grade 1, slight (0-16 points); Grade 2, mild (18-36 points); Grade 3, moderate (38-56 points); Grade 4, severe (58-76 points); and Grade 5, catastrophic (78-100 points)(Peter H. Wilson, et al., Tinnitus Reaction Questionnnaire, Journal of Speech, Language, and Hearing Research, February 1991, Vol. 34, 197-201.). The higher the score, the greater the tinnitus symptoms. Therefore, a decrease in the total score or on individual item scores indicates improvement.

In some embodiments, after the treatment the patient experiences a substantial reduction of tinnitus that is characterized by at least about a 10% decline in total Tinnitus Reaction Questionnaire (TRQ) value compared to prior to the treatment. In some embodiments, the reduction of tinnitus is characterized by a decline in total TRQ value ranging from about 10% to about 100%, for example, about 10%, about 20%, about 30%, about 40%, about 50%, about 60%, about 70%, about 80%, about 90%, and about 100%, compared to prior to the treatment.

In some embodiments, after the treatment the patient experiences a substantial reduction of tinnitus that is characterized by at least a two-point decline in total TRQ value compared to prior to the treatment. In some embodiments, the reduction of tinnitus is characterized by a decline in total TRQ value ranging from about two point to about eighty points, for example, about two points, about four points, about six points, about eight points, about ten points, about twelve points, about fourteen points, about sixteen points, about eighteen points, about twenty points, about twenty-two points, about twenty-four points, about twenty-six points, about twenty-eight points, about thirty points, about thirty-two points, about thirty-four points, about thirty-six points, about thirty-eight points, about forty points, about forty-two points, about forty-four points, about forty-six points, about forty-eight points, about fifty points, about fifty-two points, about fifty-four points, about fifty-six points, about fifty-eight points, about sixty points, about sixty-two points, about sixty-four points, about sixty-six points, about sixty-eight points, about seventy points, about seventy-two points, about seventy-four points, about seventy-six points, about seventy-eight points, or about eighty points.

In some embodiments, the reduction of tinnitus is characterized by a decline in total TRQ value of about two points. In some embodiments, the reduction of tinnitus is characterized by a decline in total TRQ value of about four points. In some embodiments, the reduction of tinnitus is characterized by a decline total TRQ value of about six points. In some embodiments, the reduction of tinnitus is characterized by a decline in total TRQ value of about eight points. In some embodiments, the reduction of tinnitus is characterized by a decline total TRQ value of about ten points. In some embodiments, the reduction of tinnitus is characterized by a decline in total TRQ value of about twelve points. In some embodiments, the reduction of tinnitus is characterized by a decline total TRQ value of about fourteen points. In some embodiments, the reduction of tinnitus is characterized by a decline in total TRQ value of about sixteen points. In some embodiments, the reduction of tinnitus is characterized by a decline total TRQ value of about eighteen points. In some embodiments, the reduction of tinnitus is characterized by a decline in total TRQ value of about twenty points. In some embodiments, the reduction of tinnitus is characterized by a decline in total TRQ value of about twenty-two points. In some embodiments, the reduction of tinnitus is characterized by a decline in total TRQ value of about twenty-four points. In some embodiments, the reduction of tinnitus is characterized by a decline in total TRQ value of about twenty-six points. In some embodiments, the reduction of tinnitus is characterized by a decline in total TRQ value of about twenty-eight points. In some embodiments, the reduction of tinnitus is characterized by a decline in total TRQ value of about thirty points. In some embodiments, the reduction of tinnitus is characterized by a decline in total TRQ value of about thirty-two points. In some embodiments, the reduction of tinnitus is characterized by a decline in total TRQ value of about thirty-four points. In some embodiments, the reduction of tinnitus is characterized by a decline in total TRQ value of about thirty-six points. In some embodiments, the reduction of tinnitus is characterized by a decline in total TRQ value of about thirty-eight points. In some embodiments, the reduction of tinnitus is characterized by a decline in total TRQ value of about forty points. In some embodiments, the reduction of tinnitus is characterized by a decline in total TRQ value of about forty-two points. In some embodiments, the reduction of tinnitus is characterized by a decline in total TRQ value of about forty-four points. In some embodiments, the reduction of tinnitus is characterized by a decline in total TRQ value of about forty-six points. In some embodiments, the reduction of tinnitus is characterized by a decline in total TRQ value of about forty-eight points. In some embodiments, the reduction of tinnitus is characterized by a decline in total TRQ value of about fifty points. In some embodiments, the reduction of tinnitus is characterized by a decline in total TRQ value of about fifty-two points. In some embodiments, the reduction of tinnitus is characterized by a decline in total TRQ value of about fifty-four points. In some embodiments, the reduction of tinnitus is characterized by a decline in total TRQ value of about fifty-six points. In some embodiments, the reduction of tinnitus is characterized by a decline in total TRQ value of about fifty-eight points. In some embodiments, the reduction of tinnitus is characterized by a decline in total TRQ value of about sixty points. In some embodiments, the reduction of tinnitus is characterized by a decline in total TRQ value of about sixty-two points. In some embodiments, the reduction of tinnitus is characterized by a decline in total TRQ value of about sixty-four points. In some embodiments, the reduction of tinnitus is characterized by a decline in total TRQ value of about sixty-six points. In some embodiments, the reduction of tinnitus is characterized by a decline in total TRQ value of about sixty-eight points. In some embodiments, the reduction of tinnitus is characterized by a decline in total TRQ value of about seventy points. In some embodiments, the reduction of tinnitus is characterized by a decline in total TRQ value of about seventy-two points. In some embodiments, the reduction of tinnitus is characterized by a decline in total TRQ value of about seventy-four points. In some embodiments, the reduction of tinnitus is characterized by a decline in total TRQ value of about seventy-six points. In some embodiments, the reduction of tinnitus is characterized by a decline in total TRQ value of about seventy-eight points. In some embodiments, the reduction of tinnitus is characterized by a decline in total TRQ value of about eighty points.

In some embodiments, after the treatment the patient experiences a substantial reduction of tinnitus that is characterized by at least a one-category change in TRQ severity classification compared to prior to the treatment. In some embodiments, the reduction of tinnitus is characterized by a one-category change TRQ severity classification compared to prior to the treatment. In some embodiments, the reduction of tinnitus is characterized by a two-category change TRQ severity classification compared to prior to the treatment. In some embodiments, the reduction of tinnitus is characterized by a three-category change TRQ severity classification compared to prior to the treatment. In some embodiments, the reduction of tinnitus is characterized by a four-category change TRQ severity classification compared to prior to the treatment.

The Tinnitus Functional Index (TFI) is a tinnitus rating scale consisting of 25 items scored on an 11-category rating scale where each category is assigned a point value with a possible total score of 100 points (Mary B. Meikle, et al., The Tinnitus Functional Index: Development of a New Clinical Measure for Chronic, Intrusive Tinnitus, Ear and Hearing, 2012, March-April; 33(2):153-76.). The total TFI score is calculated by: (1) summing all valid answers from both TFI pages (maximum possible score=250 if the respondent were to rate all 25 TFI items at the maximum value of 10); (2) dividing by the number of questions for which that respondent provided valid answers (yields the respondent's mean item score for all items having valid answers); and (3) multiplying by 10 (provides that respondent's overall TFI score within 0-100 range).

The TFI is divided into eight subscales: Intrusive (unpleasantness, intrusiveness, persistence), Sense of Control (reduced sense of control), Cognitive (cognitive interference), Sleep (sleep disturbance), Auditory (auditory difficulties attributed to tinnitus), Relaxation (interference with relaxation). Quality Of Life (quality of life reduced) and Emotional (emotional distress). The score for each subscale is determined by (the highest score for each Factor is 100): (1) Summing all of that respondent's valid answers for a given subscale; (2) Dividing by the number of valid answers that were provided by that respondent for that subscale; and (3) Multiplying by 10.

In some embodiments, after the treatment the patient experiences a substantial reduction of tinnitus that is characterized by an at least about a 10% decline in total Tinnitus Functional Index (TFI) value, or in the value of at least one of the TFI subscales, compared to prior to the treatment. In some embodiments, the reduction of tinnitus is characterized by a decline in total TFI value, or in the value of one or more of the TFI subscales, ranging from about 10% to about 100%, for example, about 10%, about 20%, about 30%, about 40%, about 50%, about 60%, about 70%, about 80%, about 90%, and about 100%, compared to prior to the treatment.

In some embodiments, after the treatment the patient experiences a substantial reduction of tinnitus that is characterized by at least a two point decline in total TFI value, or in the value of one or more of the TFI subscales, compared to prior to the treatment. In some embodiments, the reduction of tinnitus is characterized by a decline in total TFI value, or in the value of one or more of the TFI subscales, value ranging from about two point to about eighty points, for example, about two points, about four points, about six points, about eight points, about ten points, about twelve points, about fourteen points, about sixteen points, about eighteen points, about twenty points, about twenty-two points, about twenty-four points, about twenty-six points, about twenty-eight points, about thirty points, about thirty-two points, about thirty-four points, about thirty-six points, about thirty-eight points, about forty points, about forty-two points, about forty-four points, about forty-six points, about forty-eight points, about fifty points, about fifty-two points, about fifty-four points, about fifty-six points, about fifty-eight points, about sixty points, about sixty-two points, about sixty-four points, about sixty-six points, about sixty-eight points, about seventy points, about seventy-two points, about seventy-four points, about seventy-six points, about seventy-eight points, or about eighty points.

In some embodiments, the reduction of tinnitus is characterized by a decline in total TFI value, or in the value of one or more of the TFI subscales, of about two points. In some embodiments, the reduction of tinnitus is characterized by a decline in total TFI value, or in the value of one or more of the TFI subscales, of about four points. In some embodiments, the reduction of tinnitus is characterized by a decline total TFI value, or in the value of one or more of the TFI subscales, of about six points. In some embodiments, the reduction of tinnitus is characterized by a decline in total TFI value, or in the value of one or more of the TFI subscales, of about eight points. In some embodiments, the reduction of tinnitus is characterized by a decline total TFI value, or in the value of one or more of the TFI subscales, of about ten points. In some embodiments, the reduction of tinnitus is characterized by a decline in total TFI value, or in the value of one or more of the TFI subscales, of about twelve points. In some embodiments, the reduction of tinnitus is characterized by a decline total TFI value, or in the value of one or more of the TFI subscales, of about fourteen points. In some embodiments, the reduction of tinnitus is characterized by a decline in total TFI value, or in the value of one or more of the TFI subscales, of about sixteen points. In some embodiments, the reduction of tinnitus is characterized by a decline total TFI value, or in the value of one or more of the TFI subscales, of about eighteen points. In some embodiments, the reduction of tinnitus is characterized by a decline in total TFI value, or in the value of one or more of the TFI subscales, of about twenty points. In some embodiments, the reduction of tinnitus is characterized by a decline in total TFI value, or in the value of one or more of the TFI subscales, of about twenty-two points. In some embodiments, the reduction of tinnitus is characterized by a decline in total TFI value, or in the value of one or more of the TFI subscales, of about twenty-four points. In some embodiments, the reduction of tinnitus is characterized by a decline in total TFI value, or in the value of one or more of the TFI subscales, of about twenty-six points. In some embodiments, the reduction of tinnitus is characterized by a decline in total TFI value, or in the value of one or more of the TFI subscales, of about twenty-eight points. In some embodiments, the reduction of tinnitus is characterized by a decline in total TFI value, or in the value of one or more of the TFI subscales, of about thirty points. In some embodiments, the reduction of tinnitus is characterized by a decline in total TFI value, or in the value of one or more of the TFI subscales, of about thirty-two points. In some embodiments, the reduction of tinnitus is characterized by a decline in total TFI value, or in the value of one or more of the TFI subscales, of about thirty-four points. In some embodiments, the reduction of tinnitus is characterized by a decline in total TFI value, or in the value of one or more of the TFI subscales, of about thirty-six points. In some embodiments, the reduction of tinnitus is characterized by a decline in total TFI value, or in the value of one or more of the TFI subscales, of about thirty-eight points. In some embodiments, the reduction of tinnitus is characterized by a decline in total TFI value, or in the value of one or more of the TFI subscales, of about forty points. In some embodiments, the reduction of tinnitus is characterized by a decline in total TFI value, or in the value of one or more of the TFI subscales, of about forty-two points. In some embodiments, the reduction of tinnitus is characterized by a decline in total TFI value, or in the value of one or more of the TFI subscales, of about forty-four points. In some embodiments, the reduction of tinnitus is characterized by a decline in total TFI value, or in the value of one or more of the TFI subscales, of about forty-six points. In some embodiments, the reduction of tinnitus is characterized by a decline in total TFI value, or in the value of one or more of the TFI subscales, of about forty-eight points. In some embodiments, the reduction of tinnitus is characterized by a decline in total TFI value, or in the value of one or more of the TFI subscales, of about fifty points. In some embodiments, the reduction of tinnitus is characterized by a decline in total TFI value, or in the value of one or more of the TFI subscales, of about fifty-two points. In some embodiments, the reduction of tinnitus is characterized by a decline in total TFI value, or in the value of one or more of the TFI subscales, of about fifty-four points. In some embodiments, the reduction of tinnitus is characterized by a decline in total TFI value, or in the value of one or more of the TFI subscales, of about fifty-six points. In some embodiments, the reduction of tinnitus is characterized by a decline in total TFI value, or in the value of one or more of the TFI subscales, of about fifty-eight points. In some embodiments, the reduction of tinnitus is characterized by a decline in total TFI value, or in the value of one or more of the TFI subscales, of about sixty points. In some embodiments, the reduction of tinnitus is characterized by a decline in total TFI value, or in the value of one or more of the TFI subscales, of about sixty-two points. In some embodiments, the reduction of tinnitus is characterized by a decline in total TFI value, or in the value of one or more of the TFI subscales, of about sixty-four points. In some embodiments, the reduction of tinnitus is characterized by a decline in total TFI value, or in the value of one or more of the TFI subscales, of about sixty-six points. In some embodiments, the reduction of tinnitus is characterized by a decline in total TFI value, or in the value of one or more of the TFI subscales, of about sixty-eight points. In some embodiments, the reduction of tinnitus is characterized by a decline in total TFI value, or in the value of one or more of the TFI subscales, of about seventy points. In some embodiments, the reduction of tinnitus is characterized by a decline in total TFI value, or in the value of one or more of the TFI subscales, of about seventy-two points. In some embodiments, the reduction of tinnitus is characterized by a decline in total TFI value, or in the value of one or more of the TFI subscales, of about seventy-four points. In some embodiments, the reduction of tinnitus is characterized by a decline in total TFI value, or in the value of one or more of the TFI subscales, of about seventy-six points. In some embodiments, the reduction of tinnitus is characterized by a decline in total TFI value, or in the value of one or more of the TFI subscales, of about seventy-eight points. In some embodiments, the reduction of tinnitus is characterized by a decline in total TFI value, or in the value of one or more of the TFI subscales, of about eighty points.

Methods of Treating Child and Adolescent Depression, Child and Adolescent Anxiety and Child and Adolescent Suicidal Ideation and Behavior

In some embodiments, the present disclosure provides methods of treating child and adolescent depression comprising administering an effective amount of etifoxine or a pharmaceutically acceptable salt thereof. In some embodiments, the patient is treated during puberty. In some embodiments, the patient is treated during menarche or menarche transition. In some embodiments, the patient is treated during spermarche or spermarche transition.

In some embodiments, the present disclosure provides methods of treating child and adolescent suicidal ideation and behavior comprising administering an effective amount of etifoxine or a pharmaceutically acceptable salt thereof. In some embodiments, the patient is treated during puberty. In some embodiments, the patient is treated during menarche or menarche transition. In some embodiments, the patient is treated during spermarche or spermarche transition.

In some embodiments, the present disclosure provides methods of treating child and adolescent anxiety comprising administering an effective amount of etifoxine or a pharmaceutically acceptable salt thereof. In some embodiments, the patient is treated during puberty. In some embodiments, the patient is treated during menarche or menarche transition. In some embodiments, the patient is treated during spermarche or spermarche transition.

In some embodiments, the present methods employ etifoxine, or a pharmaceutically acceptable salt thereof, as the sole active ingredient used to treat child and adolescent depression, child and adolescent suicidal ideation and behavior, and child and adolescent anxiety. In some embodiments, the present methods employ etifoxine, or a pharmaceutically acceptable salt thereof, in conjunction with one or more active ingredients used to treat child and adolescent depression, child and adolescent suicidal ideation and behavior, and child and adolescent anxiety. In some embodiments, etifoxine or a pharmaceutically acceptable salt thereof is administered in combination with an additional active ingredient used to treat child and adolescent depression, child and adolescent suicidal ideation and behavior, and child and adolescent anxiety, e.g., co-formulated or administered separately.

Suicide is the second leading cause of death in adolescents worldwide (Colich, N. L., et al., HPA-axis reactivity interacts with stage of pubertal development to predict the onset of depression. Psychoneuroendocrinology 55, 94-101 (2015) and Gunnar, M. R., et al., Stressor paradigms in developmental studies: What does and does not work to produce mean increases in salivary cortisol. Psychoneuroendocrinology 34, 953-967 (2009).). Child and Adolescent or pediatric (ages 5-18) major depressive disorder (“MDD”) and suicide ideation and attempts are increasing, with reports in the U.S. of pediatric suicidal attempts and ideation visits to the emergency room increasing from 580,000 to 1.12 million (or 92.1%) from 2007 to 2015 (Rice, F. et a. Adolescent and adult differences in major depression symptom profiles. J Affect Disorders 243, 175-181 (2019).). Thus, there is a need for therapies for the treatment of child and adolescent depression as well for child and adolescent suicidal ideation and behavior.

Children and adolescent females post-menarche have a 50% greater risk of developing depression and anxiety relative to pre-menarche individuals (Patton et al., Menarche and the onset of depression and anxiety in Victoria, Australia. Journal of Epidemiology and Community Health 1996; 50:661-666).

DSM-5 Diagnostic criteria is similar for child, adolescent, and adult depressive disorder with the exception that DSM-5 recognizes irritability, rather than depressed mood, as a core symptom for child and adolescent depression (Burstein, B., et al., Suicidal Attempts and Ideation Among Children and Adolescents in US Emergency Departments, 2007-2015. Jana Pediatr 173, (2019) and Guerry, J., et al., In Search of HPA Axis Dysregulation in Child and Adolescent Depression. Clin Child Fam Psych 14, 135-160 (2011).).

Presentation of depression in adolescence differs from adulthood in that vegetative symptoms (appetite and weight change, loss of energy and insomnia) are more common in adolescent MDD, while anhedonia, loss of interest and concentration issues are more typical in adults with MDD (Colich).

Clinical data from recent birth cohorts suggest that adolescence is the most common period of onset of the first episode of depression (Miller, A., et al., Adolescent Suicide as a Failure of Acute Stress-Response Systems. Annu Rev Clin Psycho 15, 1-26 (2019).).

Hypothalamic pituitary and adrenal (HPA) axis is a central regulator to human responses to stress and HPA axis dysregulation precedes and predicts depression in children and adolescents (Miller and Burstein).

Activation of the HPA axis involves an increase in the synthesis and release of neuroactive derivative of steroid hormones. This peripheral increase in neuroactive steroids, such as allopregnanolone, contributes to the fine control of the HPA axis and response to stress (Colich).

Neurosteroids modulate the HPA axis by controlling the activity of GABAergic neurons that are involved in the regulation of corticotropin hormone and gonadotropin hormone release through phasic and tonic GABAergic inhibition, which is mediated by both synaptic and extrasynaptic GABA-A receptors (Burstein).

Hormonal sensitivity in girls is reported to predict their development of MDD, for instance in early pubertal development onset of MDD was predicted by lower reactivity to cortisol, a stress hormone, while late pubertal development onset of MDD was predicted by higher reactivity (American Psychiatric Association. Diagnostic and statistical manual of mental disorders (5th ed.).).

According to some embodiments of the present disclosure, the dosing frequency and dose amount per administration of etifoxine are selected to provide therapeutic effects for the treatment of child and adolescent depression. In some embodiments, the dosing frequency and dose amount per administration of etifoxine are selected to provide therapeutic effects for the treatment of child and adolescent depression during puberty. In some embodiments, the dosing frequency and dose amount per administration of etifoxine are selected to provide therapeutic effects for the treatment of child and adolescent depression during menarche or menarche transition. In some embodiments, the dosing frequency and dose amount per administration of etifoxine are selected to provide therapeutic effects for the treatment of child and adolescent depression during spermarche or spermarche transition.

Reduction child and adolescent depression can be determined by various methods. In some embodiments, the effectiveness of a dosage regimen can be determined by evaluation via Children's Depression Rating Scale, Revised (CDRS-R) value. In some embodiments, the effectiveness of a dosage regimen can be determined by evaluation via Montgomery-Åsberg Depression Rating Scale (MADRS) value. In yet some other embodiments, the effectiveness of a dosage regimen can be determined by evaluation via CDRS-R value, MADRS value, [add other provided by Praxis] or any combination thereof.

The Children's Depression Rating Scale (CDRS-R) is a 17-item scale (each rated 1 to 5 or 1 to 7) that has been widely used for evaluation of children and adolescents with depression. The highest possible score is 113 (the most severe measure of depression), and the lowest is 17 (not suffering from depression). A score of ≥40 is indicative of depression, whereas a score ≤28 is used to define remission (minimal or no symptoms). CDRS-R is based on a semi-structured interview with the child (or an adult informant who knows the child well).

In some embodiments, after the treatment the patient experiences a substantial reduction of child and adolescent depression that is characterized by at least about a 30% decline in CDRS-R value compared to prior to the treatment. In some embodiments, the reduction of child and adolescent depression is characterized by a decline in CDRS-R value ranging from about 30% to about 100%, for example, about 30%, about 40%, about 50%, about 60%, about 70%, about 80%, about 90%, and about 100%, compared to prior to the treatment.

In some embodiments, after the treatment the patient experiences a substantial reduction of child and adolescent depression that is characterized by at least a one point decline in CDRS-R value compared to prior to the treatment. In some embodiments, the reduction of child and adolescent depression is characterized by a decline in CDRS-R value ranging from about one point to about five points, for example, about one point, about two points, about three points, about four points, and about five points compared to prior to the treatment. In some embodiments, the reduction of child and adolescent depression is characterized by a decline CDRS-R value of about two points. In some embodiments, the reduction of child and adolescent depression is characterized by a decline in CDRS-R value of about three points. In some embodiments, the reduction of child and adolescent depression is characterized by a decline in CDRS-R value of about four points. In some embodiments, the reduction of child and adolescent depression is characterized by a decline in CDRS-R value of about five points. In certain embodiments, the reduction of child and adolescent depression is characterized by remission according to CDRS-R value after said treatment (i.e., CDRS-R value of 28 or less).

The Montgomery Åsberg Depression Rating Scale (MADRS) is a depression rating scale consisting of 10 items, each rated 0 to 6. The 10 items represent the core symptoms of depressive illness. The total score of the 10 items ranges from 0 to 60. Decrease in the total score or on individual items indicates improvement (Montgomery S. A. and Åsberg M. A. New Depression Scale Designed to be Sensitive to Change, Br. J. Psychiatry. (1979) April; 134, pages 382-9.).

In some embodiments, after the treatment the patient experiences a substantial reduction of child and adolescent depression that is characterized by at least about a 30% decline in Montgomery Åsberg Depression Rating Scale (MADRS) value compared to prior to the treatment. In some embodiments, the reduction of child and adolescent depression is characterized by a decline in MADRS value ranging from about 30% to about 100%, for example, about 30%, about 40%, about 50%, about 60%, about 70%, about 80%, about 90%, and about 100%, compared to prior to the treatment.

In some embodiments, after the treatment the patient experiences a substantial reduction of child and adolescent depression that is characterized by at least a one point decline in MADRS value compared to prior to the treatment. In some embodiments, the reduction of child and adolescent depression is characterized by a decline in MADRS value ranging from about one point to about five points, for example, about one point, about two points, about three points, about four points, and about five points compared to prior to the treatment. In some embodiments, the reduction of child and adolescent depression is characterized by a decline MADRS value of about two points. In some embodiments, the reduction of child and adolescent depression is characterized by a decline in MADRS value of about three points. In some embodiments, the reduction of child and adolescent depression is characterized by a decline in MADRS value of about four points. In some embodiments, the reduction of child and adolescent depression is characterized by a decline in MADRS value of about five points. In certain embodiments, the reduction of child and adolescent depression is characterized by remission according to MADRS value after said treatment (i.e., MADRS value of 12 or less).

The HAM-D is a depression rating scale consisting of 17 items, eight items are scored on a 5-point scale (ranging from 0 to 4), and 9 items are scores on a 3-point scale (ranging from 0 to 2). The total score of the 17 items ranges from 0 to 50 with higher scores indicating greater depression. The total score the 17 items is used to categorize the severity of depression: normal (total score between 0 and 7), mild depression (total score between 8 and 13), moderate depression (total score between 14-18), severe depression (total score between 19-22). Therefore, a decrease in the total score or on individual item scores indicates improvement Hamilton, M. A Rating Scale for Depression, Journal of Neurology, Neurosurgery, and Psychiatry. (1960) 23, pages 56-62.

In some embodiments, after the treatment the patient experiences a substantial reduction of child and adolescent depression that is characterized by at least about a 30% decline in total Hamilton Depression Rating Scale (HAM-D) value compared to prior to the treatment. In some embodiments, the reduction of child and adolescent depression is characterized by a decline in HAM-D value ranging from about 30% to about 100%, for example, about 30%, about 40%, about 50%, about 60%, about 70%, about 80%, about 90%, and about 100%, compared to prior to the treatment.

In some embodiments, after the treatment the patient experiences a substantial reduction of child and adolescent depression that is characterized by at least a one point decline in HAM-D value compared to prior to the treatment. In some embodiments, the reduction of child and adolescent depression is characterized by a decline in HAM-D value ranging from about one point to about twenty points, for example, about one point, about two points, about three points, about four points, about five points, about six points, about seven points, about eight points, about nine points, about ten points, about eleven points, about twelve points, about thirteen points, about fourteen points, about fifteen points, about sixteen points, about seventeen points, about eighteen points, about nineteen points, and about twenty points compared to prior to the treatment. In some embodiments, the reduction of child and adolescent depression is characterized by a decline HAM-D value of about two points. In some embodiments, the reduction of child and adolescent depression is characterized by a decline in HAM-D value of about three points. In some embodiments, the reduction of child and adolescent depression is characterized by a decline in HAM-D value of about four points. In some embodiments, the reduction of child and adolescent depression is characterized by a decline in HAM-D value of about five points. In some embodiments, the reduction of child and adolescent depression is characterized by a decline HAM-D value of about six points. In some embodiments, the reduction of child and adolescent depression is characterized by a decline in HAM-D value of about seven points. In some embodiments, the reduction of child and adolescent depression is characterized by a decline in HAM-D value of about eight points. In some embodiments, the reduction of child and adolescent depression is characterized by a decline in HAM-D value of about nine points. In some embodiments, the reduction of child and adolescent depression is characterized by a decline HAM-D value of about ten points. In some embodiments, the reduction of child and adolescent depression is characterized by a decline in HAM-D value of about eleven points. In some embodiments, the reduction of child and adolescent depression is characterized by a decline in HAM-D value of about twelve points. In some embodiments, the reduction of child and adolescent depression is characterized by a decline in HAM-D value of about thirteen points. In some embodiments, the reduction of child and adolescent depression is characterized by a decline HAM-D value of about fourteen points. In some embodiments, the reduction of child and adolescent depression is characterized by a decline in HAM-D value of about fifteen points. In some embodiments, the reduction of child and adolescent depression is characterized by a decline in HAM-D value of about sixteen points. In some embodiments, the reduction of child and adolescent depression is characterized by a decline in HAM-D value of about seventeen points. In some embodiments, the reduction of child and adolescent depression is characterized by a decline HAM-D value of about eighteen points. In some embodiments, the reduction of child and adolescent depression is characterized by a decline in HAM-D value of about nineteen points. In some embodiments, the reduction of child and adolescent depression is characterized by a decline in HAM-D value of about twenty points.

In some embodiments, after the treatment the patient experiences a substantial reduction of child and adolescent depression that is characterized by at least a one category change in HAM-D severity classification compared to prior to the treatment. In some embodiments, the reduction of child and adolescent depression is characterized by a one category change HAM-D severity classification compared to prior to the treatment. In some embodiments, the reduction of child and adolescent depression is characterized by a two category change HAM-D severity classification compared to prior to the treatment. In some embodiments, the reduction of child and adolescent depression is characterized by a three category change HAM-D severity classification compared to prior to the treatment. In certain embodiments, the reduction of child and adolescent depression is characterized by remission according to HAM-D value after said treatment (i.e., total HAM-D value of 7 or less).

The Hamilton Rating Scale for Anxiety (HAM-A) is an anxiety rating scale consisting of 14 items that assess anxious mood, tension, fear, insomnia, intellectual (cognitive) functioning, depressed mood, behavior at interview, somatic (sensory), cardiovascular, respiratory, gastrointestinal, genitourinary, autonomic, and somatic (muscular) symptom (Hamilton, M. The Assessment of Anxiety States by Rating, Br J Med Psychol. (1959); 32(1), pages 50-5). Each symptom is rated from 0 (absent) to 4 (maximum severity) scale. The total score is used to categorize the severity of anxiety: mild severity (total score less than 17), mild to moderate severity (total score between 18-24), and moderate to severe (total score between 25-30). Total scores range from 0 to 56 with higher scores indicating greater severity.

In some embodiments, after the treatment the patient experiences a substantial reduction of child and adolescent depression that is characterized by at least about a 30% decline in total HAM-A value compared to prior to the treatment. In some embodiments, the reduction of anxiety is characterized by a decline in HAM-A value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of child and adolescent depression that is characterized by partial remission of the patient's depression. In some embodiments, after the treatment the patient experiences a substantial reduction of MDD that is characterized by partial remission of the patient's depression. In certain embodiments, partial remission of MDD is where the symptoms of the immediately previous major depressive episode are present, but full criteria are not met, or there is a period lasting less than 2 months without any significant symptoms of a major depressive episode following the end of such an episode (i.e., the DSM-5's definition of partial remission).

In some embodiments, after the treatment the patient experiences a substantial reduction of child and adolescent depression that is characterized by full remission of the patient's depression. In some embodiments, after the treatment the patient experiences a substantial reduction of MDD that is characterized by full remission of the patient's depression. In certain embodiments, full remission is where during the past 2 months, no significant signs or symptoms of the disturbance were present (i.e., the DSM-5's definition of full remission).

The Clinical Global Impression (CGI) (Guy 1976 (Guy W (1976), ECDEU Assessment Manual for Psychopharmacology, Revised. Rockville, Md.: US Department of Health, Education and Welfare) consists of three subscales: the CGI-Severity (CGI-S), the CGI-Improvement (CGI-I) and Efficacy Index. The CGI-S assesses the clinician's impression of the patient's current mental illness. A treating clinician categorizes the severity of the patient's current mental illness on a 7-point scale: 1 (normal, not at all ill), 2 (borderline mentally ill), 3 (mildly ill), 4 (moderately ill), 5 (markedly ill), 6 (severely ill), and 7 (among the most extremely ill patients). The CGI-I assesses the participant's improvement (or worsening) from baseline. A treating clinician categorizes the patient's condition relative to Baseline (e.g., prior to administering an antidepressant) on a 7-point scale: 1 (very much improved), 2 (much improved), 3 (minimally improved), 4 (no change), 5 (minimally worse), 6 (much worse), and 7 (very much worse).

In some embodiments, after the treatment the patient experiences a substantial reduction of child and adolescent depression that is characterized by at least a one point decline in CGI-S value compared to prior to the treatment. In some embodiments, the reduction of child and adolescent depression is characterized by a one point decline in CGI-S value compared to prior to the treatment. In some embodiments, the reduction of child and adolescent depression is characterized by a two point decline in CGI-S value compared to prior to the treatment. In some embodiments, the reduction of child and adolescent depression is characterized by a three point decline in CGI-S value compared to prior to the treatment.

In some embodiments, after the treatment the patient experiences a substantial reduction of child and adolescent depression that is characterized by at least a one point decline in CGI-I value compared to prior to the treatment. In some embodiments, the reduction of child and adolescent depression is characterized by a one point decline in CGI-I value compared to prior to the treatment. In some embodiments, the reduction of child and adolescent depression is characterized by a two point decline in CGI-I value compared to prior to the treatment. In some embodiments, the reduction of child and adolescent depression is characterized by a three point decline in CGI-I value compared to prior to the treatment.

The Symptoms of Depression Questionnaire (SDQ) is a 44-item, self-report scale that consists of five subscales: SDQ-1, SDQ-2, SDQ-3, SDQ-4 and SDQ-5. SDQ-1 includes items related to lassitude, mood, and cognitive functioning. SDQ-2 includes items related to anxiety, agitation, irritability, and anger. SDQ-3 includes items related to suicidal ideation. SDQ-4 assesses disruptions in sleep quality. SDQ-5 includes items on changes in appetite and weight. SDQ is used to assess symptom severity across several subtypes of depression (Pedrelli, P., et al., Reliability and Validity of the Symptoms of Depression Questionnaire (SDQ), CNS Spectr. 2014 December; 19(6), pages 535-546.). The items are rated on a 6-point scale. Each item is rated based on a participant's perception of what is normal for the individual (score=2), what is better than normal (score=1), and what is worse than normal (scores=3-6). Total scores range from 0 to 264 with higher scores indicating greater severity.

In some embodiments, after the treatment the patient experiences a substantial reduction of child and adolescent depression that is characterized by at least about a 10% decline in total SDQ scale value compared to prior to the treatment. In some embodiments, the reduction of child and adolescent depression is characterized by a decline in total SDQ scale value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of child and adolescent depression that is characterized by at least about a 10% decline in at least one subscale value selected from SDQ-1, SDQ-2, SDQ-3, SDQ-4 and SDQ-5 compared to prior to the treatment. In some embodiments, the reduction of child and adolescent depression is characterized by a decline in at least one subscale value selected from SDQ-1, SDQ-2, SDQ-3, SDQ-4 and SDQ-5 value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of child and adolescent depression that is characterized by at least a one point decline in Global PSQI (described above) score compared to prior to the treatment. In some embodiments, the reduction of child and adolescent depression is characterized by a one point decline in Global PSQI score compared to prior to the treatment. In some embodiments, the reduction of child and adolescent depression is characterized by a two point decline in Global PSQI score compared to prior to the treatment. In some embodiments, the reduction of child and adolescent depression is characterized by a three point decline in Global PSQI score compared to prior to the treatment.

According to some embodiments of the present disclosure, the dosing frequency and dose amount per administration of etifoxine are selected to provide therapeutic effects for the treatment of child and adolescent suicidal ideation and behavior. In some embodiments, the dosing frequency and dose amount per administration of etifoxine are selected to provide therapeutic effects for the treatment of child and adolescent suicidal ideation and behavior during puberty. In some embodiments, the dosing frequency and dose amount per administration of etifoxine are selected to provide therapeutic effects for the treatment of child and adolescent suicidal ideation and behavior during menarche or menarche transition. In some embodiments, the dosing frequency and dose amount per administration of etifoxine are selected to provide therapeutic effects for the treatment of child and adolescent suicidal ideation and behavior during spermarche or spermarche transition.

Reduction child and adolescent suicidal ideation and behavior can be determined by various methods. In some embodiments, the effectiveness of a dosage regimen can be determined by evaluation via Columbia-Suicide Severity Rating Scale (C-SSRS) value. In some embodiments, the effectiveness of a dosage regimen can be determined by evaluation via Beck Scale for Suicidal Ideation (BSSI) value. In some embodiments, the effectiveness of a dosage regimen can be determined by evaluation via the suicidality item of the Montgomery-Åsberg Depression Rating Scale (MADRS-SI) value. In yet some other embodiments, the effectiveness of a dosage regimen can be determined by evaluation via C-SSRS value, BSSI value, MADRS-SI, or any combination thereof.

The Columbia-Suicide Severity Rating Scale (C-SSRS) value rates an individual's degree of suicidal ideation (SI) on a scale, ranging from “wish to be dead” to “active suicidal ideation with specific plan and intent” where each question is answered yes or no. The scale identifies Suicidal Ideation severity and intensity, which may be indicative of an individual's intent to commit suicide. C-SSRS Suicidal Ideation severity subscale ranges from 0 (no Suicidal Ideation) to 5 (active Suicidal Ideation with plan and intent).

In some embodiments, after the treatment the patient experiences a substantial reduction of child and adolescent suicidal ideation and behavior that is characterized by an increase of at least one in the number of “no” responses in the Suicidal Ideation subscale of the C-SSRS compared to prior to the treatment. In some embodiments, after the treatment the patient experiences a substantial reduction of child and adolescent suicidal ideation and behavior that is characterized by an increase of at least two in the number of “no” responses in the Suicidal Ideation subscale of the C-SSRS compared to prior to the treatment. In some embodiments, after the treatment the patient experiences a substantial reduction of child and adolescent suicidal ideation and behavior that is characterized by an increase of at least three in the number of “no” responses in the Suicidal Ideation subscale of the C-SSRS compared to prior to the treatment. In some embodiments, after the treatment the patient experiences a substantial reduction of child and adolescent suicidal ideation and behavior that is characterized by an increase of at least four in the number of “no” responses in the Suicidal Ideation subscale of the C-SSRS compared to prior to the treatment.

In some embodiments, after the treatment the patient experiences a substantial reduction of child and adolescent suicidal ideation and behavior that is characterized by an increase of one in the number of “no” responses in the Suicidal Ideation subscale of the C-SSRS compared to prior to the treatment. In some embodiments, after the treatment the patient experiences a substantial reduction of child and adolescent suicidal ideation and behavior that is characterized by an increase of two in the number of “no” responses in the Suicidal Ideation subscale of the C-SSRS compared to prior to the treatment. In some embodiments, after the treatment the patient experiences a substantial reduction of child and adolescent suicidal ideation and behavior that is characterized by an increase of three in the number of “no” responses in the Suicidal Ideation subscale of the C-SSRS compared to prior to the treatment. In some embodiments, after the treatment the patient experiences a substantial reduction of child and adolescent suicidal ideation and behavior that is characterized by an increase of four in the number of “no” responses in the Suicidal Ideation subscale of the C-SSRS compared to prior to the treatment.

In some embodiments, after the treatment the patient experiences a substantial reduction of child and adolescent suicidal ideation and behavior that is characterized by at least a one point decline in C-SSRS Suicidal Ideation severity subscale value compared to prior to the treatment. In some embodiments, the reduction of child and adolescent suicidal ideation and behavior is characterized by a decline in C-SSRS Suicidal Ideation severity subscale value ranging from about one point to about five points, for example, about one point, about two points, about three points, about four points, and about five points compared to prior to the treatment. In some embodiments, the child and adolescent suicidal ideation and behavior is characterized by a decline C-SSRS Suicidal Ideation severity subscale value of about two points. In some embodiments, the reduction of child and adolescent suicidal ideation and behavior is characterized by a decline C-SSRS Suicidal Ideation severity subscale value of about three points. In some embodiments, the reduction of child and adolescent suicidal ideation and behavior is characterized by a decline in C-SSRS Suicidal Ideation severity subscale value of about four points.

The Beck Scale for Suicidal Ideation (BSSI) is a 21-item self or clinician administered instrument used to measure the current intensity of patients' specific attitudes, behaviors and plans to commit suicide. Scores range 0-42, with higher score indicating higher intensity.

In some embodiments, after the treatment the patient experiences a substantial reduction of child and adolescent suicidal ideation and behavior that is characterized by at least about a 30% decline in Beck Scale for Suicidal Ideation (BSSI) value compared to prior to the treatment. In some embodiments, the reduction of child and adolescent suicidal ideation and behavior is characterized by a decline in BSSI value ranging from about 30% to about 100%, for example, about 30%, about 40%, about 50%, about 60%, about 70%, about 80%, about 90%, and about 100%, compared to prior to the treatment.

In some embodiments, after the treatment the patient experiences a substantial reduction of child and adolescent suicidal ideation and behavior that is characterized by at least a one point decline in BSSI value compared to prior to the treatment. In some embodiments, the reduction of child and adolescent suicidal ideation and behavior is characterized by a decline in BSSI value ranging from about one point to about five points, for example, about one point, about two points, about three points, about four points, and about five points compared to prior to the treatment. In some embodiments, the reduction of child and adolescent suicidal ideation and behavior is characterized by a decline BSSI value of about two points. In some embodiments, the reduction of child and adolescent suicidal ideation and behavior is characterized by a decline in BSSI value of about three points. In some embodiments, the reduction of child and adolescent suicidal ideation and behavior is characterized by a decline in BSSI value of about four points. In some embodiments, the reduction of child and adolescent suicidal ideation and behavior is characterized by a decline in BSSI value of about five points.

The Suicidality Item of the MADRS (MADRS-SI) is one item from MADRS and the ranges from 0 to 6. A score of 2 corresponds to fleeting, passive suicidal ideation; a score of 4 indicates that suicidal ideation is frequent with at least moderate intensity but without specific plans or intention; and a score of 6 corresponds to active intention and planning for suicide.

In some embodiments, after the treatment the patient experiences a substantial reduction of child and adolescent suicidal ideation and behavior that is characterized by at least a one point decline in MADRS-SI value compared to prior to the treatment. In some embodiments, the reduction of child and adolescent suicidal ideation and behavior is characterized by a decline in MADRS-SI value ranging from about one point to about five points, for example, about one point, about two points, about three points, about four points, and about five points compared to prior to the treatment. In some embodiments, the child and adolescent suicidal ideation and behavior is characterized by a decline MADRS-SI value of about two points. In some embodiments, the reduction of child and adolescent suicidal ideation and behavior is characterized by a decline MADRS-SI value of about three points. In some embodiments, the reduction of child and adolescent suicidal ideation and behavior is characterized by a decline in MADRS-SI value of about four points. In some embodiments, the reduction of child and adolescent suicidal ideation and behavior is characterized by a decline in MADRS-SI value of about five points.

In certain embodiments, the dosing frequency and dose amount per administration of Etifoxine are selected to provide therapeutic effects for the treatment of MDD that is partially responsive to other antidepressant therapies. In certain embodiments, the MDD patient that is partially responsive to other antidepressant therapies is an adult patient meeting DSM-IV criteria for MDD who had had an inadequate response to prior antidepressant therapy (1 to 3 courses) in the current episode and who had demonstrated an inadequate response to 8 weeks of prospective antidepressant therapy. Inadequate response for prospective treatment is defined as less than 50% improvement on the 17-item version of the Hamilton Depression Rating Scale (HAM-D), minimal HAM-D score of 14, and a Clinical Global Impressions Improvement rating of no better than minimal improvement. Inadequate response to prior treatment is defined as less than 50% improvement as perceived by the patient after a minimum of 6 weeks of antidepressant therapy at or above the minimal effective dose. In certain embodiments, the dosing frequency and dose amount per administration of Etifoxine are selected to provide therapeutic effects for the treatment of moderate MDD that is partially responsive to other antidepressant therapies. In certain embodiments, the dosing frequency and dose amount per administration of Etifoxine are selected to provide therapeutic effects for the treatment of severe MDD that is partially responsive to other antidepressant therapies. In certain embodiments, the dosing frequency and dose amount per administration of Etifoxine are selected to provide therapeutic effects for the treatment of MDD that is partially responsive to treatment with SSRIs. In certain embodiments, the dosing frequency and dose amount per administration of Etifoxine are selected to provide therapeutic effects for the treatment of moderate MDD that is partially responsive to treatment with SSRIs. In certain embodiments, the dosing frequency and dose amount per administration of Etifoxine are selected to provide therapeutic effects for the treatment of severe MDD that is partially responsive to treatment with SSRIs.

According to some embodiments, the substantial reduction in child and adolescent depression provided by the methods of the present disclosure requires treatment for a specified time interval (e.g., at least one week) before the patient experiences substantial reduction of child and adolescent depression (i.e., there is an induction period before the patient experiences a substantial reduction in depression). In some embodiments, after treatment for at least one week, at least two weeks, at least three weeks, at least four weeks, at least five weeks, at least six weeks, at least seven weeks or at least eight weeks, the patient experiences a substantial reduction of child and adolescent depression compared to prior to the treatment. In certain embodiments, after treatment for at least one week the patient experiences a substantial reduction of child and adolescent depression compared to prior to the treatment. According to this embodiment, the substantial reduction in depression may be expressed using any of the methods described herein (for example, decline in total Hamilton Depression Rating Scale (HAM-D) value compared to prior to the treatment, improvement in the Montgomery Åsberg Depression Rating Scale value compared to prior to the treatment, etc.).

According to some embodiments of the present disclosure, the dosing frequency and dose amount per administration of etifoxine are selected to provide therapeutic effects for the treatment of child and adolescent anxiety. In some embodiments, the dosing frequency and dose amount per administration of etifoxine are selected to provide therapeutic effects for the treatment of child and adolescent anxiety. In some embodiments, the dosing frequency and dose amount per administration of etifoxine are selected to provide therapeutic effects for the treatment of child and adolescent anxiety during menarche or menarche transition. In some embodiments, the dosing frequency and dose amount per administration of etifoxine are selected to provide therapeutic effects for the treatment of child and adolescent anxiety during spermarche or spermarche transition.

Reduction child and adolescent anxiety can be determined by various methods. In some embodiments, the effectiveness of a dosage regimen can be determined by evaluation via HAM-A value.

In some embodiments, after the treatment the patient experiences a substantial reduction of child and adolescent anxiety that is characterized by at least a one point decline in HAM-A value compared to prior to the treatment. In some embodiments, the reduction of child and adolescent anxiety is characterized by a decline in HAM-A value ranging from about one point to about five points, for example, about one point, about two points, about three points, about four points, and about five points compared to prior to the treatment. In some embodiments, the reduction of child and adolescent anxiety is characterized by a decline HAM-A value of about two points. In some embodiments, the reduction of child and adolescent anxiety is characterized by a decline in HAM-A value of about three points. In some embodiments, the reduction of child and adolescent anxiety is characterized by a decline in HAM-A value of about four points. In some embodiments, the reduction of child and adolescent anxiety is characterized by a decline in HAM-A value of about five points.

In some embodiments, after the treatment the patient experiences a substantial reduction of child and adolescent anxiety that is characterized by at least a one category change in HAM-A severity classification compared to prior to the treatment. In some embodiments, the reduction of child and adolescent anxiety is characterized by a one category change HAM-A severity classification compared to prior to the treatment. In some embodiments, the reduction of child and adolescent anxiety is characterized by a two category change HAM-A severity classification compared to prior to the treatment. In some embodiments, the reduction of depression is characterized by a three category change HAM-A severity classification compared to prior to the treatment.

In some embodiments, after the treatment the patient experiences a substantial reduction of child and adolescent depression that is characterized by at least about a 30% decline in total HAM-A value compared to prior to the treatment. In some embodiments, the reduction of child and adolescent anxiety is characterized by a decline in HAM-A value ranging from about 10% to about 100%, for example, about 20%, about 30%, about 40%, about 50%, about 60%, about 70%, about 80%, about 90%, and about 100%, compared to prior to the treatment.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

What is claimed is:

1. A method of treating tinnitus comprising administering to a patient in need thereof a therapeutically effective amount of etifoxine or a pharmaceutically acceptable salt thereof.

2. The method of claim 1, wherein the tinnitus is caused by an otologic condition, a neurologic condition or an infectious condition.

3. The method of claim 1, wherein the tinnitus is drug-induced tinnitus.

4. The method of claim 1, wherein the tinnitus is selected from the group consisting of acute peripheral tinnitus, chronic tinnitus and chronic subjective tinnitus.

5. The method of any one of claims 1-4, wherein after the administering, the patient experiences a substantial reduction in tinnitus compared to prior to said administering.

6. The method of any one of claims 1-5, wherein after the administering, the patient experiences a reduction of tinnitus that is characterized by an at least five point decline in total Tinnitus Handicap Inventory (THI) value compared to prior to the treatment.

7. The method of any one of claims 1-6, wherein after the administering, the patient experiences a reduction of tinnitus that is characterized by an at least 20% reduction in THI value compared to prior to the treatment.

8. The method of any one of claims 1-7, wherein after the administering, the patient experiences a reduction of tinnitus that is characterized by an at least one-category change in THI severity classification compared to prior to the treatment.

9. The method of any one of claims 1-8, wherein after the administering, the patient experiences a reduction of tinnitus that is characterized by an at least five point decline in total Tinnitus Handicap Inventory (THQ) value compared to prior to the treatment.

10. The method of any one of claims 1-9, wherein after the administering, the patient experiences a reduction of tinnitus that is characterized by an at least 20% reduction in THQ value compared to prior to the treatment.

11. The method of any one of claims 1-10, wherein after the administering, the patient experiences a reduction of tinnitus that is characterized by an at least five point decline in total Tinnitus Reaction Questionnaire (TRQ) value compared to prior to the treatment.

12. The method of any one of claims 1-11, wherein after the administering, the patient experiences a reduction of tinnitus that is characterized by an at least 20% reduction in total TRQ value compared to prior to the treatment.

13. The method of any one of claims 1-12, wherein after the administering, the patient experiences a reduction of tinnitus that is characterized by an at least one-category change in TRQ severity classification compared to prior to the treatment.

14. The method of any one of claims 1-13, wherein after the administering, the patient experiences a reduction of tinnitus that is characterized by an at least five point decline in total Tinnitus Functional Index (TFI) value compared to prior to the treatment.

15. The method of any one of claims 1-14, wherein after the administering, the patient experiences a reduction of tinnitus that is characterized by an at least 20% reduction in TFI value compared to prior to the treatment.

16. A method of treating dyspnea comprising administering to a patient in need thereof a therapeutically effective amount of etifoxine or a pharmaceutically acceptable salt thereof.

17. The method of claim 16, wherein the dyspnea is selected from the group consisting of dyspnea in asthma, dyspnea in chronic obstructive pulmonary disorder (COPD), dyspnea in idiopathic pulmonary fibrosis, dyspnea in heart disease (such as congestive heart failure), dyspnea in gastroesophageal reflux disease (GERD) (GERD), dyspnea in cancer and dyspnea in other severe respiratory disorders.

18. The method of any one of claims 16-17, wherein after the administering, the patient experiences a substantial reduction in dyspnea compared to prior to said administering.

19. The method of any one of claims 16-18, wherein after the administering, the patient experiences a reduction of dyspnea that is characterized by an at least a 1.0 point reduction in the EXACT-Respiratory Symptoms (E-RSTM) breathlessness subscale score compared to prior to the treatment.

20. The method of any one of claims 16-19, wherein after the administering, the patient experiences a reduction of dyspnea that is characterized by an at least a one point change in the Borg dyspnea scale value total score compared to prior to the treatment.

21. The method of any one of claims 16-20, wherein after the administering, the patient experiences a reduction of dyspnea that is characterized by an at least a one point change in any of the Borg dyspnea scale domains (sensory-perceptual, affective distress or symptom impact) compared to prior to the treatment.

22. The method of any one of claims 16-21, wherein after the administering, the patient experiences a reduction of dyspnea that is characterized by an at least one category change in the Modified Medical Research Council Scale compared to prior to the treatment.

23. The method of any one of claims 16-22, wherein after the administering, the patient experiences a reduction of dyspnea that is characterized by an at least one category change in at least one of the 7 questions of the PROMIS Pool v 1.0 Dyspnea Emotional Response Scale compared to prior to the treatment.

24. The method of any one of claims 16-23, wherein after the administering, the patient experiences a reduction of dyspnea that is characterized by an at least a one category change in at least one of the 10 questions of the PROMIS Item Bank v1.0 Dyspnea Severity-Short Form 10a Scale compared to prior to the treatment.

25. The method of any one of claims 16-24, wherein after the administering, the patient experiences a reduction of dyspnea that is characterized by an at least a one category change in at least one of the 4 items or a one category change in the question “I have been short of breath” of the PROMIS Item Bank v1.0 Dyspnea Characteristics Scale compared to prior to the treatment.

26. The method of any one of claims 16-25, wherein after the administering, the patient experiences a reduction of dyspnea that is characterized by an at least two point decline in total Hamilton Rating Scale for anxiety (HAM-A) value compared to prior to the treatment.

27. The method of any one of claims 16-26, wherein after the administering, the patient experiences a reduction of dyspnea that is characterized by an at least 50% reduction in HAM-A value compared to prior to the treatment.

28. The method of any one of claims 16-27, wherein after the administering, the patient experiences a reduction of dyspnea that is characterized by an at least one category change in HAM-A severity classification compared to prior to the treatment.

29. The method of any one of claims 16-28, wherein after the administering, the patient experiences a reduction of dyspnea that is characterized by at least about a 20% decline in total GAD-7 value compared to prior to the treatment.

30. The method of any one of claims 16-29, wherein after the administering, the patient experiences a reduction of dyspnea that is characterized by an at least two point decline in total GAD-7 value compared to prior to the treatment.

31. The method of any one of claims 16-30, wherein after the administering, the patient experiences a reduction of dyspnea that is characterized by an at least one category change in GAD-7 severity classification compared to prior to the treatment.

32. The method of any one of claims 16-31, wherein the etifoxine is administered as an adjunctive to an opioid.

33. A method of treating a substance addiction disorder comprising administering to a patient in need thereof a therapeutically effective amount of etifoxine or a pharmaceutically acceptable salt thereof.

34. The method of claim 32, wherein the substance addiction disorder is an opioid use disorder.

35. The method of claim 32, wherein the substance addiction disorder is a cocaine use disorder.

36. The method of claim 32, wherein the substance addiction disorder is alcohol use disorder.

37. The method of claim 32, wherein the substance addiction disorder is benzodiazepine use disorder.

38. The method of any one of claims 33-37, wherein after the administering, the patient experiences a substantial reduction in substance addiction disorder compared to prior to said administering.

39. The method of any one of claims 33-34, wherein after the administering, the patient experiences a reduction of opioid use disorder that is characterized by abstinence to opioid use during the period of etifoxine administration.

40. The method of any one of claims 33-34 and 39, wherein after the administering, the patient experiences a reduction of opioid use disorder that is characterized by a statistically significant decrease in the percentage of opioid-free weeks in the etifoxine treated group compared to a placebo treated group during the period of etifoxine administration.

41. The method of any one of claims 33-34 and 39-40, wherein after the administering, the patient experiences a reduction of opioid use disorder that is characterized by substantial improvement in craving assessment compared to prior to the treatment.

42. The method of any one of claims 33-34 and 39-41, wherein after the administering, the patient experiences a reduction of opioid use disorder that is characterized by a statistically significant change in retention assessment compared to a placebo treated group.

43. The method of any one of claims 33-34 and 39-42, wherein after the administering, the patient experiences a reduction of opioid use disorder that is characterized by a significant statistical difference in the mean change in number of days of retention in etifoxine treatment group relative to placebo.

44. The method of any one of claims 33-43, wherein the etifoxine is administered as an adjunctive to methadone: buprenorphine; buprenorphine and naloxone; naltrexone, benzodiazepine, lofexidine, medically supervised withdrawal (detoxification), residential rehabilitation treatment, mutual help groups or outpatient substance use disorder services (e.g., counseling or medication for addiction), or combinations thereof.

45. A method of treating child and adolescent depression comprising administering to a patient in need thereof a therapeutically effective amount of etifoxine or a pharmaceutically acceptable salt thereof.

46. The method of claim 45, wherein the patient experiences a reduction of child and adolescent depression that is characterized by at least about a 30% decline in Children's Depression Rating Scale (CDRS-R) value compared to prior to the treatment.

47. The method of any one of claims 45-46, wherein the patient experiences a reduction of child and adolescent depression that is characterized by at least a one point decline in CDRS-R value compared to prior to the treatment.

48. The method of any one of claims 45-47, wherein the patient experiences a reduction of child and adolescent depression that is characterized by at least about a 30% decline in Montgomery Åsberg Depression Rating Scale (MADRS) value compared to prior to the treatment.

49. The method of any one of claims 45-48, wherein the patient experiences a reduction of child and adolescent depression that is characterized by at least a one point decline in MADRS value compared to prior to the treatment.

50. A method of treating child and adolescent suicidal ideation and behavior comprising administering to a patient in need thereof a therapeutically effective amount of etifoxine or a pharmaceutically acceptable salt thereof.

51. The method of claim 50, wherein after said administering, the patient experiences a reduction of child and adolescent suicidal ideation and behavior that is characterized by an increase of at least one in the number of “no” responses in the Suicidal Ideation subscale of the C-SSRS compared to prior to the treatment.

52. The method of any one of claims 50-51, wherein after said administering, the patient experiences a reduction of child and adolescent suicidal ideation and behavior that is characterized by at least a one point decline in C-SSRS Suicidal Ideation severity subscale value compared to prior to the treatment.

53. The method of any one of claims 50-52, wherein after said administering, the patient experiences a reduction of child and adolescent suicidal ideation and behavior that is characterized by at least about a 30% decline in Beck Scale for Suicidal Ideation (BSSI) value compared to prior to the treatment.

54. The method of any one of claims 50-53, wherein after said administering, the patient experiences a reduction of child and adolescent suicidal ideation and behavior that is characterized by at least a one point decline in BSSI value compared to prior to the treatment.

55. The method of any one of claims 50-54, wherein after said administering, the patient experiences a reduction of child and adolescent suicidal ideation and behavior that is characterized by at least a one point decline in MADRS-SI value compared to prior to the treatment.

56. A method of treating child and adolescent anxiety comprising administering to a patient in need thereof a therapeutically effective amount of etifoxine or a pharmaceutically acceptable salt thereof.

57. The method of claim 56, wherein after said administering, the patient experiences a reduction of anxiety that is characterized by an at least two point decline in total Hamilton Rating Scale for anxiety (HAM-A) value compared to prior to the treatment.

58. The method of any one of claims 56-57, wherein after said administering, the patient experiences a reduction of anxiety that is characterized by an at least 50% reduction in HAM-A value compared to prior to the treatment.

59. The method any one of claims 56-58, wherein after said administering, the patient experiences a reduction of anxiety that is characterized by an at least one category change in HAM-A severity classification compared to prior to the treatment.

60. The method of any one of claims 45-59, wherein the patient is treated during puberty.

61. The method of any one of claims 45-59, wherein the patient is treated during menarche or menarche transition.

62. The method of any one of claims 45-59, wherein the patient is treated during spermarche or spermarche transition.

63. The method of any one of claims 1-62, wherein the total administered daily dose of etifoxine or a pharmaceutically acceptable salt thereof is between about 5 mg-250 mg.

64. The method of any one of claims 1-63, wherein the etifoxine or a pharmaceutically acceptable salt thereof is orally administered for at least one week.

65. The method of any one of claims 1-64, wherein the etifoxine or a pharmaceutically acceptable salt thereof is orally administered for at least one month.

66. The method of any one of claims 1-65, wherein the etifoxine or a pharmaceutically acceptable salt thereof is administered once per day.

67. The method of any one of claims 1-65, wherein the etifoxine or a pharmaceutically acceptable salt thereof is administered twice per day.

68. The method of any one of claims 1-65, wherein the etifoxine or a pharmaceutically acceptable salt thereof is administered three times per day.

69. The method of any one of claims 1-31, 32-43 and 45-62, wherein the etifoxine is administered as a monotherapy.

70. The method of any one of claims 1-69, wherein the etifoxine or a pharmaceutically acceptable salt thereof is administered in a capsule.

71. The method of any one of claims 1-69, wherein the etifoxine or a pharmaceutically acceptable salt thereof is administered in a tablet.
